(19) United States
(12) Patent Application Publication
(10) Pub. No.: US 2025/0136708 A1
(43) Pub. Date: May 1, 2025

(54) ANTI-HUMAN ATP6V1B2 ANTIBODIES AND USES THEREOF

(71) Applicant: YEDA RESEARCH AND DEVELOPMENT CO. LTD., Rehovot (IL)

(72) Inventors: Valery KRIZHANOVSKY, Rehovot (IL); Nataly FREIZUS, Rehovot (IL); Dganit BAR, Ness Ziona (IL); Vered CARMON, Petah Tiqwa (IL)

(73) Assignee: YEDA RESEARCH AND DEVELOPMENT CO. LTD., Rehovot (IL)

(21) Appl. No.: 18/833,064

(22) PCT Filed: Feb. 6, 2023
(86) PCT No.: PCT/IL2023/050128

§ 371 Date: Jul. 25, 2024

Related U.S. Application Data
(60) Provisional application No. 63/307,119, filed on Feb. 6, 2022.

(51) Int. Cl. C07K 16/28; C07K 16/40

(52) U.S. Cl. CPC C07K 16/2896; C07K 16/40; C07K 2317/33; C07K 2317/41; C07K 2317/732

(57) ABSTRACT

Described herein are anti-human ATP6VIB2 antibodies. The antibodies can be used to target senescent cells. Thus, the anti-ATP6VIB2 antibodies would be useful in treating diseases and conditions associated with cellular senescence.

### FIELD OF INTEREST

The present disclosure relates in general to the field of antibodies and uses thereof. More specifically, the present disclosure provides anti-human ATP6V1B2 monoclonal antibodies and uses thereof.

### SEQUENCE LISTING STATEMENT

The instant application contains a Sequence Listing which has been submitted electronically in XML format and is hereby incorporated by reference in its entirety. Said XML copy, created on Jan. 9, 2023, is named P-611141-PC_SQL_09JAN23.xml and is 42.9 kilo bytes in size.

### BACKGROUND

The multisubunit vacuolar-type proton pump (H⁺- or V-ATPase) is essential for acidification of diverse intracellular compartments in eukaryotic cells. These include endosomes, lysosomes, clathrin-coated vesicles, secretory vesicles, chromaffin granules, and the central vacuoles of plants and fungi. H⁺-ATPases are also found at high density in the plasma membrane of certain specialized cell types such as renal intercalated cells, neutrophils, osteoclasts, and some cells in the male genital tract, where they play important roles in urinary acidification, cytoplasmic pH homeostasis, bone resorption, and sperm maturation, respectively.

V-ATPase-dependent organelle acidification is necessary for intracellular processes such as protein sorting, zymogen activation, receptor-mediated endocytosis, and synaptic vesicle proton gradient generation. The lysosomal acidification caused by activated v-ATPase enables activation of a set of lysosome-resident peptidases that facilitate the degradation of proteins delivered to the lysosome. It has been demonstrated that autophagosome-lysosome fusion can proceed in the absence of v-ATPase, but the resulting neutral lysosomal pH is unlikely to support the generation of sufficient free amino acids for active cell growth.

The general structure of H⁺-ATPases comprises two functional sectors, V1 and V0. The cytoplasmic V1 domain binds and hydrolyzes ATP, providing the energy for H+ translocation across the transmembrane V0 domain. A structural model, based mostly on topology studies of the yeast and bovine clathrin-coated vesicle H⁺-ATPases, suggests that there are at least 13 different subunits: the V₁ domain (640 kDa) comprises subunits A-H, in a proposed stoichiometry of A₃B₃C₁D₁E₁F₁G₂H₁, while the V₀ domain (260 kDa) contains five subunits.

The precise function(s) of many of the proton pump's subunits and the interactions between them remain undetermined. Moreover, in higher eukaryotes, several H+-ATPase subunits have recently been shown to have multiple isoforms encoded by different genes and with differing tissue expression patterns. The existence of different subunit isoforms may play an important role in the localization and activity of proton pumps in specific cell types and subcellular compartments.

ATP6V1B2 is one of the two V1 domain B subunit isoforms, and as it is highly expressed in the organ of cerebrum and in the organelle of lysosome, it is also called a brain isoform or lysosomal V1 subunit B2. It is also highly expressed in osteoclasts, kidney, and many other tissues. It has been reported that de novo mutation in ATP6V1B2 impairs lysosome acidification and causes dominant deafness. In all of the instances above, the localization of ATP6V1B2 was identified as intracellular.

Using high-throughput proteomics analysis ATP6V1B2 polypeptide was identified as expressed on cell surface of senescent cells (WO/2016/185481).

### SUMMARY

Described herein in one aspect is an isolated anti-ATP6V1B2 antibody, wherein the antibody comprises a heavy chain variable region (VH) comprising complementarity determining region 1 (HCDR1), HCDR2 and HCDR3, and a light chain variable region (VL) having complementarity determining region 1 (LCDR1), LCDR2 and LCDR3, wherein the HCDR1, HCDR2 and HCDR3 and the LCDR1, LCDR2 and LCDR3 for the antibody comprise the amino acid sequences of (i) SEQ ID NOs: 2, 4, and 6, respectively, and SEQ ID NO: 9, amino acids KVS, and SEQ ID NO: 13, respectively; or (ii) SEQ ID NOs: 2, 4, and 6, respectively, and SEQ ID NO: 20, amino acids KVS, and SEQ ID NO: 13 respectively; or (iii) SEQ ID NOs: 24, 26, and 6, respectively, and SEQ ID NO: 29, amino acids KVS and SEQ ID NO: 31, respectively.

In some embodiments, the anti-ATP6V1B2 antibody disclosed herein can be an IgG, a Fv, a scFv, a Fab, or a F(ab′)2. In some embodiments, the amino acid sequence of the heavy chain variable region comprises one or more humanized framework (FR) sequences, and the amino acid sequence of the light chain variable region comprises one or more humanized FR sequences.

In another aspect, the present disclosure provides a composition comprising the anti-ATP6V1B2 antibody disclosed herein and a pharmaceutically acceptable carrier.

In another aspect, the present disclosure provides a nucleic acid construct comprising one or more nucleic acid sequences that encode one or more of a light chain variable region, or a heavy chain variable region, or fragments thereof of the anti-ATP6V1B2 antibody disclosed herein. In some embodiments, the present disclosure provides an expression vector comprising such nucleic acid construct. In some embodiments, the present disclosure provides a host cell comprising such expression vector.

In another aspect, described herein are methods of treating a disease in a subject, comprising the step of administering to the subject a composition comprising an effective amount of the anti-ATP6V1B2 antibody, wherein: (a) the anti-ATP6V1B2 antibody further comprises a heavy chain fragment crystallizable region (Fc region), wherein said Fc region comprises at least one amino acid residue substitution comprising S239D, 1332E, A330L, G236A, H268F, S324T, S267E, or any combination thereof; or wherein fucosylation of the Fc region is reduced in comparison to a Fc region of an anti-ATP6V1B2 antibody produced in the presence of fucose; or both; or (b) the anti-ATP6V1B2 antibody further comprises an ATP6V1B2 antibody-drug conjugate; or (c) both (a) and (b). In a related aspect, described herein are methods of treating a disease in a subject, comprising the step of administering to the subject a composition comprising an effective amount of the anti-ATP6V1B2 antibody, wherein: (a) the anti-ATP6V1B2 antibody further comprises a heavy chain fragment crystallizable region (Fc region), wherein said Fc region comprises two amino acid residue substitutions comprising S239D and 1332E and wherein fucosylation of the Fc region is reduced in comparison to a Fc region of an anti-ATP6V1B2 antibody produced in the presence of fucose; or (b) the anti-ATP6V1B2 antibody further comprises an ATP6V1B2 antibody-drug conjugate; or (c) both (a) and (b). In some embodiments, the disease or condition to be treated is associated with cellular senescence in a subject. In a related aspect, the disease or condition associated with cellular senescence is an age-related disease or condition. In another related aspect, the age-related disease comprises a fibrotic disease or condition, an inflammatory disease or condition, or a therapy induced senescence in cancer.

### BRIEF DESCRIPTION OF THE DRAWINGS

The subject matter is particularly pointed out and distinctly claimed in the concluding portion of the specification. The anti-human ATP6V1B2 antibodies disclosed in detail herein, features thereof and uses thereof, however, both as to organization and method of operation, together with objects, features, and advantages thereof, may best be understood by reference to the following detailed description when read with the accompanying drawings in which:

FIG. 1 presents a flow chart providing an overview of the steps performed to produce and analyze the anti-human ATP6V1B2 monoclonal antibodies.

FIG. 2 shows three anti-ATP6V1B2 antibodies (2G05, 2E11 and 4G06) binding to human recombinant protein. Clones were tested as short chain variable fragments (scFv) using ELISA.

FIGS. 3A-3C show that three anti-ATP6V1B2 antibodies (2G05, 2E11 and 4G06) preferentially bind the surface protein expressed in senescent human lung fibroblasts (IMR-90). Surface binding was examined in non-permeabilized cells using FACS. FIG. 3A: antibody 2G05.

FIG. 3B: antibody 2E11. FIG. 3C: antibody 4G06.

FIGS. 4A-4C show that three anti-ATP6V1B2 antibodies (2G05, 2E11 and 4G06) preferentially bind the surface protein expressed in senescent mouse lung fibroblasts (CCL-206). Surface binding was examined in non-permeabilized cells using FACS. FIG. 4A: antibody 2G05.

FIG. 4B: antibody 2E11. FIG. 4C: antibody 4G06.

FIGS. 5A-5B show that two anti-ATP6V1B2 antibodies (FIG. 5A: antibody 2G05;

FIG. 5B: antibody 2E11) bind to senescent IPF (Idiopathic Pulmonary Fibrosis) lung fibroblasts. Senescence was evaluated using SA β-gal staining and antibody binding to surface ATP6V1B2 was measured in non-permeabilized cells using FACS. Results are presented as percent positive cells±SE.

FIGS. 6A-6B show that two anti-ATP6V1B2 antibodies (2G05, 2E11) bind to senescent A549 lung adenocarcinoma cells but not to control non-senescent A549 cells. Senescence was induced by 100 uM doxorubicin. FIG. 6A: senescence was confirmed by SA-β-gal staining. FIG. 6B: Antibody binding to surface ATP6V1B2 in senescent and non-senescent (growing) cells was measured in non-permeabilized cells using FACS. Results are presented as percent positive cells±SE.

FIGS. 7A-7C show binding of the three anti-ATP6V1B2 antibodies (2G05, 2E11 and 4G06) to senescent IMR-90 or CCL-206 cells as compared with three commercially available anti-ATP6V1B2 antibodies. FIG. 7A presents a list of commercially available anti-ATP6V1B2 antibodies. FIG. 7B shows binding to senescent IMR-90 cells by the indicated antibodies. FIG. 7C shows binding to senescent CCL-206 cells by the indicated antibodies.

FIGS. 8A-8B show the efficacy of two anti-ATP6V1B2 antibodies (FIG. 8A: antibody 2G05; FIG. 8B: antibody 2E11) in activating the ADCC (Antibody Dependent Cellular Cytotoxicity) pathway. The antibodies were tested at increasing concentrations using the mFcγRIV ADCC Reporter Bioassay (Promega M1211), and senescent CCL-206 cells were used as target cells. Antibodies 2G05 and 2E11 are formatted as IgG2a comprising two substation mutations in the Fc region (S239D and 1332E) and were produced in the absence of fucose.

FIG. 9 shows two anti-ATP6V1B2 antibodies (2G05, 4G06) have cross reactivity with ATP6V1B1. The antibodies were tested as short chain variable fragments (scFv) using ELISA.

### DETAILED DESCRIPTION

In the following detailed description, numerous specific details are set forth in order to provide a thorough understanding of the anti-ATP6V1B2 antibodies disclosed herein. However, it will be understood by those skilled in the art that preparation and uses of antibodies disclosed herein may in certain cases be practiced without these specific details. In other instances, well-known methods, procedures, and components have not been described in detail so as not to obscure the disclosure presented herein.

Throughout this application, various references or publications are cited. Disclosures of these references or publications in their entireties are hereby incorporated by reference into this application in order to more fully describe the state of the art.

As used herein, the term “antibody” may be used interchangeably with the term “immunoglobulin”, having all the same qualities and meanings. An antibody binding domain or an antigen binding site can be a fragment of an antibody or a genetically engineered product of one or more fragments of the antibody, which fragment is involved in specifically binding with a target antigen. In some embodiments, an antibody comprises an antigen binding fragment of an antibody.

By “specifically binding” is meant that the binding is selective for the antigen of interest and can be discriminated from unwanted or nonspecific interactions. For example, in some embodiments, an antibody is said to specifically bind an ATP6V1B2 epitope when the equilibrium dissociation constant is ≤10⁻⁵, 10⁻⁶, or 10⁻⁷ M. In some embodiments, the equilibrium dissociation constant may be ≤10⁻⁸ M or 10⁻⁹ M. In some further embodiments, the equilibrium dissociation constant may be ≤10⁻¹⁰ M, 10⁻¹¹ M, or 10⁻¹² M. In some embodiments, the equilibrium dissociation constant may be in the range of ≤10⁻⁵ M to 10⁻¹² M.

As used herein, the term “antibody” encompasses an antibody fragment or fragments that retain binding specificity including, but not limited to, IgG, heavy chain variable region (VH), light chain variable region (VL), Fab fragments, F(ab′)2 fragments, scFv fragments, Fv fragments, a nanobody, minibodies, diabodies, triabodies, tetrabodies, and single domain antibodies (see, e.g., Hudson and Souriau, Nature Med. 9:129-134 (2003)).

A skilled artisan would appreciate that the term “antibody” encompasses not only intact polyclonal or monoclonal antibodies, but also humanized antibodies. In some embodiments, anti-ATP6V1B2 antibodies disclosed herein encompass humanized or primatized antibodies as these terms are generally understood in the art.

As used herein, the term “heavy chain variable region” may be used interchangeably with the term “VH domain” or the term “VH”, having all the same meanings and qualities. As used herein, the term “light chain variable region” may be used interchangeably with the term “VL domain” or the term “VL”, having all the same meanings and qualities. A skilled artisan would recognize that a “heavy chain variable region” or “VH” with regard to an antibody encompasses the fragment of the heavy chain that contains three complementarity determining regions (CDRs) interposed between flanking stretches known as framework regions. The framework regions (FR) are more highly conserved than the CDRs, and form a scaffold to support the CDRs. Similarly, a skilled artisan would also recognize that a “light chain variable region” or “VL” with regard to an antibody encompasses the fragment of the light chain that contains three CDRs interposed between framework regions.

As used herein, the term “complementarity determining region” or “CDR” refers to the hypervariable region(s) of a heavy or light chain variable region. Proceeding from the N-terminus, each of a heavy or light chain polypeptide has three CDRs denoted as “CDR1,” “CDR2,” and “CDR3”. Crystallographic analysis of a number of antigen-antibody complexes has demonstrated that the amino acid residues of CDRs form extensive contact with a bound antigen, wherein the most extensive antigen contact is with the heavy chain CDR3. Thus, the CDR regions are primarily responsible for the specificity of an antigen-binding site. In one embodiment, an antigen-binding site includes six CDRs, comprising the CDRs from each of a heavy and a light chain variable region.

As used herein, the term “framework region” or “FR” refers to the four flanking amino acid sequences which frame the CDRs of a heavy or light chain variable region. Some FR residues may contact the bound antigen; however, FR residues are primarily responsible for folding the variable region into the antigen-binding site. In some embodiments, the FR residues responsible for folding the variable regions comprise residues directly adjacent to the CDRs. Within FRs, certain amino residues and certain structural features are very highly conserved. In this regard, all variable region sequences contain an internal disulfide loop of around 90 amino acid residues. When a variable region folds into an antigen binding site, the CDRs are displayed as projecting loop motifs that form an antigen-binding surface. It is generally recognized that there are conserved structural regions of FR that influence the folded shape of the CDR loops into certain “canonical” structures regardless of the precise CDR amino acid sequence. Furthermore, certain FR residues are known to participate in non-covalent interdomain contacts which stabilize the interaction of the antibody heavy and light chains.

Wu and Kabat (Tai Te Wu, Elvin A. Kabat. An analysis of the sequences of the variable regions of bence jones proteins and myeloma light chains and their implications for antibody complementarity. Journal of Experimental Medicine, 132, 2, 8 (1970); Kabat EA, Wu T T, Bilofsky H, Reid-Miller M, Perry H. Sequence of proteins of immunological interest. Bethesda: National Institute of Health; 1983. 323 (1983)) pioneered the alignment of antibody peptide sequences, and their contributions in this regard were several-fold: Firstly, through study of sequence similarities between variable domains, they identified correspondent residues that to a greater or lesser extent were homologous across all antibodies in all vertebrate species, inasmuch as they adopted similar three-dimensional structure, played similar functional roles, interacted similarly with neighboring residues, and existed in similar chemical environments. Secondly, they devised a peptide sequence numbering system in which homologous immunoglobulin residues were assigned the same position number. One skilled in the art can unambiguously assign to any variable domain sequence what is now commonly called Kabat numbering without reliance on any experimental data beyond the sequence itself. Thirdly, Kabat and Wu calculated variability for each Kabat-numbered sequence position, by which is meant the finding of few or many possible amino acids when variable domain sequences are aligned. They identified three contiguous regions of high variability embedded within four less variable contiguous regions. Kabat and Wu formally demarcated residues constituting these variable tracts, and designated these “complementarity determining regions” (CDRs), referring to chemical complementarity between antibody and antigen. A role in three-dimensional folding of the variable domain, but not in antigen recognition, was ascribed to the remaining less-variable regions, which are now termed “framework regions”. Fourth, Kabat and Wu established a public database of antibody peptide and nucleic acid sequences, which continues to be maintained and is well known to those skilled in the art.

An antibody may exist in various forms or having various domains including, without limitation, a complementarity determining region (CDR), a variable region (Fv), a VH domain, a VL domain, a single chain variable region (scFv), and a Fab fragment.

A person of ordinary skill in the art would appreciate that a scFv is a fusion polypeptide comprising the variable heavy chain (VH) and variable light chain (VL) regions of an immunoglobulin, connected by a short linker peptide, the linker may have, for example, 10 to about 25 amino acids.

A skilled artisan would also appreciate that the term “Fab” with regard to an antibody generally encompasses that portion of the antibody consisting of a single light chain (both variable and constant regions) bound to the variable region and first constant region of a single heavy chain by a disulfide bond, whereas F(ab′)₂ comprises a fragment of a heavy chain comprising a VH domain and a light chain comprising a VL domain.

A skilled artisan would also appreciate that the term “fragment crystallizable region” (“Fc region” or “Fc domain”) encompasses the constant region of an immunoglobulin molecule, the tail region of an antibody that interacts with cell surface receptors called Fc receptors and some proteins of the complement system. This property allows antibodies to activate the immune system. The Fc region of an antibody interacts with a number of Fc receptors and ligands, imparting an array of important functional capabilities referred to as effector functions, as described herein. For IgG the Fc region comprises Ig domains CH2 and CH3. An important family of Fc receptors for the IgG isotype are the Fc gamma receptors (FcgammaR, FcγR, FCGR). These receptors mediate communication between antibodies and the cellular arm of the immune system.

There is a relationship between the structure and composition of human IgG1 Fc chain and the antibody's effector capabilities. For example, fucose removal has been shown to enhance ADCC significantly via improved binding to Fc gamma receptors, and this property applies to antigens at various expression levels (Niwa et al. Clin. Cancer Res. 11:2327-2336). In addition, several mutations in the Fc chain have been shown to increase binding to Fc gamma receptors and complement and enhance antibody-dependent cellular cytotoxicity (ADCC) and/or complement dependent cytotoxicity (CDC) activities (Moore et al. mAbs 2:2, 181-189 www.landesbioscience.com/journals/mabs/article/11158). These mutations include H268F/S324T (FT); S267E/H268F/S324T (EFT); G236A/1332E (AE); S239D/1332E (DE); and combinations thereof (e.g., FT+DE; FT+AE; EFT+AE). Other possible combinations include, but are not limited to, A330L/S239D/1332E (LDE). (Lazer et al. [2006] Proc. Nat. Acad. Sci. vol. 103 (11): 4005-4010)

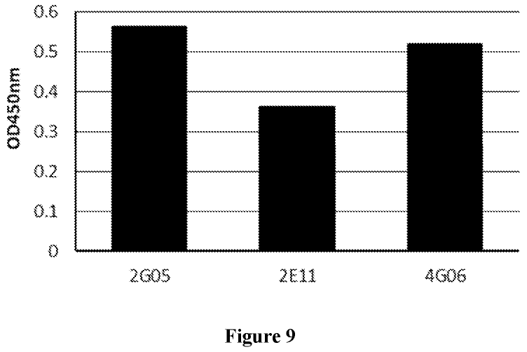

A skilled artisan would appreciate that the term “isolated antibody” may encompass an antibody that is substantially free of other antibodies having different antigenic specificities, e.g., an isolated antibody that specifically binds human ATP6V1B2 (hATP6V1B2) is substantially free of antibodies that specifically bind antigens other than hATP6V1B2. An isolated antibody that specifically binds hATP6V1B2 may, however, have cross-reactivity to other antigens, such as ATP6V1B2 molecules from other species, or with ATP6V1B1 as demonstrated in FIG. 9. Moreover, an isolated antibody may be substantially free of other cellular material and/or chemicals.

A skilled artisan would appreciate that the term “antibody-dependent cell-mediated cytotoxicity” (ADCC), also referred to as “antibody-dependent cellular cytotoxicity,” may encompass a mechanism of cell-mediated immune defense whereby an effector cell of the immune system actively lyses a target cell, whose membrane-surface antigens have been bound by specific antibodies. Typically, a Fc gamma (Fcγ) receptor (FcgammaR, FcγR or FCGR) on the surface of an immune effector cell binds to the Fc region of an antibody, which specifically binds to a target cell. When the Fc gamma receptor binds to the antibody, the Fc gamma receptor's immunoreceptor tyrosine-based activation motif (ITAM) is phosphorylated, which triggers the activation of effector cells and the secretion of various substances (lyase, perforin, granzyme, tumor necrosis factor [TNF]) that mediate target cell destruction. ADCC comprises one of the mechanisms through which antibodies, as part of the humoral immune response, can act to limit and contain infection. ADCC requires an effector cell, such as a natural killer (NK) cell that typically interacts with immunoglobulin G (IgG) antibodies. However, macrophages, monocytes, neutrophils, and eosinophils can also mediate ADCC.

A skilled artisan would appreciate that the term “complement dependent cytotoxicity” (CDC) may encompass an effector function of IgG and IgM antibodies. When they are bound to a surface antigen on target cell (e.g., a bacterial or viral infected cell or a tumor cell), the complement pathway is triggered by binding protein C1q binding to these antibodies, resulting in formation of a membrane attack complex (MAC) on the surface of target cells, leading to a classical pathway of complement activation and lysis of target cells. This system is efficiently activated by human IgG1, IgG3 and IgM antibodies.

A skilled artisan would appreciate that the term “antibody-dependent cellular phagocytosis” (ADCP) may encompass a highly regulated process in which an antibody eliminates binding target and initiates phagocytosis by linking its Fc domain to a specific receptor on the phagocytic cell (Tay et al. (2019) Front Immunol 10:332. doi: 10.3389/fimmu.2019.00332). Unlike ADCC, ADCP can mediate monocytes, macrophages, neutrophils and dendritic cells via FcγRIIa, FcγRI and FcγRIIIa, where FcγRIIa (CD32a) on macrophages represents the major pathway.

A skilled artisan would appreciate that the term “natural killer cell” (NK cell or NKC), also known as a “large granular lymphocyte” (LGL), comprises a type of cytotoxic lymphocyte critical to the innate immune system that belongs to the family of innate lymphoid cells (ILC). NK cells also play a role in the adaptive immune response. NK cells provide rapid responses to virus-infected cell and other intracellular pathogens approximately 3 days post-infection, and they also respond to tumor formation. Typically, immune cells detect the major histocompatibility complex (MHC) presented on infected cell surfaces, triggering cytokine release, causing the death of the infected cell by lysis or apoptosis. Unlike other immune cells, however, NK cells have the ability to recognize and kill stressed cells in the absence of antibodies and MHC, allowing for a much faster immune reaction. NK cells, along with macrophages and several other cell types, express the Fc receptor (FcR) molecule (FC-gamma-RIII=CD16), an activating biochemical receptor that binds the Fc portion of IgG class antibodies. This binding allows NK cells to target cells against which have undergone a humoral response and to lyse cells through ADCC. This response depends on the affinity level of the Fc receptor expressed on NK cells, which can have high, intermediate, and low affinity for the Fc portion of the antibody. This affinity level is determined by the amino acid in position 158 of the protein, which can be phenylalanine (F allele) or valine (V allele). For example, individuals with high-affinity FcRgammRIII (158 V/V allele) typically respond better to antibody therapy, including lymphoma patients who received the antibody Rituxan, with patients who expressed the 158 V/V allele having a better antitumor response (Snyder K M et al. (2018) Front Immunol 9:2873. doi: 10.3389/fimmu.2018.02873). Only 15-25% of the population expresses the 158 V/V allele.

### Anti-ATP6V1B2 Antibodies

In certain embodiments, the present disclosure provides an isolated anti-ATP6V1B2 antibody, wherein the antibody comprises a heavy chain variable region having complementarity determining regions (CDR) H-CDR1, H-CDR2, and H-CDR3. In some embodiments, the FR region around the heavy chain H-CDRs comprises the FR amino acid sequences of the mouse monoclonal from which the heavy chain CDR regions were derived. In some embodiments, the FR region around the heavy chain CDRs comprises a humanized FR amino acid sequence. In one embodiment, the present disclosure also encompasses a composition comprising the above-mentioned antibody and a pharmaceutically acceptable carrier.

In certain embodiments, the present disclosure provides an isolated anti-ATP6V1B2 antibody, wherein the antibody comprises a light chain variable region having complementarity determining regions (CDR) L-CDR1, L-CDR2, and L-CDR3. In some embodiments, the FR region around the light chain CDRs comprises the FR amino acid sequences of the mouse monoclonal from which the light chain CDR regions were derived. In some embodiments, the FR region around the light chain CDRs comprises a humanized FR amino acid sequence. In one embodiment, the present disclosure also encompasses a composition comprising the above-mentioned antibody and a pharmaceutically acceptable carrier.

In certain embodiments, the present disclosure provides an isolated anti-ATP6V1B2 antibody, wherein the antibody comprises a heavy chain variable region having complementarity determining regions (CDR) H-CDR1, H-CDR2, and H-CDR3 and a light chain variable region having complementarity determining regions (CDR) L-CDR1, L-CDR2, and L-CDR3. In some embodiments, the FR region around the heavy chain H-CDRs comprises the FR amino acid sequences of the mouse monoclonal from which the heavy chain CDR regions were derived and the FR region around the light chain CDRs comprises the FR amino acid sequences of the mouse monoclonal from which the light chain CDR regions were derived. In some embodiments, the FR region around the heavy chain CDRs comprises a humanized FR amino acid sequence and the FR region around the light chain CDRs comprises a humanized FR amino acid sequence. In one embodiment, the present disclosure also encompasses a composition comprising the above-mentioned antibody and a pharmaceutically acceptable carrier.

In certain embodiments, the present disclosure provides an isolated anti-ATP6V1B2 antibody, wherein the antibody comprises a heavy chain variable region having complementarity determining regions (CDR) H-CDR1, H-CDR2, and H-CDR3, and a light chain variable region having complementarity determining regions (CDR) L-CDR1, L-CDR2, and L-CDR3. In some embodiments, the FR region around the heavy chain and the FR region around the light chain CDRs comprise the FR amino acid sequences of the mouse monoclonal from which the heavy and light chain CDR regions were derived. In some embodiments, the FR region around the heavy chain CDRs and the FR region around the light chain CDRs comprise humanized FR amino acid sequences. In one embodiment, the present disclosure also encompasses a composition comprising the above-mentioned antibody and a pharmaceutically acceptable carrier.

In certain embodiments of an isolated anti-ATP6V1B2 antibody as disclosed herein, the antibody comprises heavy and light chain variable CDR regions sequences from one species but FR sequences of another species. Non-limiting examples of such antibodies are isolated anti-ATP6V1B2 antibodies comprising mouse H-CDR and L-CDR regions and human FR regions, for example, wherein the H-CDR and L-CDR regions are obtained from a murine monoclonal antibody to human ATP6V1B2.

In some embodiments, an isolated anti-ATP6V1B2 antibody disclosed herein comprises a heavy chain variable region having complementarity determining region 1 (H-CDR1), H-CDR2 and H-CDR3, wherein the H-CDR1, H-CDR2 and H-CDR3 comprise amino acid sequences of SEQ ID NOs: 2, 4, and 6, respectively. In some embodiments, an isolated anti-ATP6V1B2 antibody disclosed herein comprises a heavy chain variable region having complementarity determining region 1 (H-CDR1), H-CDR2 and H-CDR3, wherein the H-CDR1, H-CDR2 and H-CDR3 comprise amino acid sequences of SEQ ID NOs: 24, 26, and 6, respectively. In certain embodiments of an isolated anti-ATP6V1B2 antibody, the amino acid sequence of the heavy chain variable region comprises one or more humanized framework (FR) sequences.

In some embodiments, an isolated anti-ATP6V1B2 antibody disclosed herein comprises a light chain variable region having complementarity determining region 1 (L-CDR1), L-CDR2 and L-CDR3, wherein the L-CDR1, L-CDR2 and L-CDR3 comprise amino acid sequences of SEQ ID NO: 9, amino acids KVS and SEQ ID NO: 13, respectively. In some embodiments, an isolated anti-ATP6V1B2 antibody disclosed herein comprises a light chain variable region having complementarity determining region 1 (L-CDR1), L-CDR2 and L-CDR3, wherein the L-CDR1, L-CDR2 and L-CDR3 comprise amino acid sequences of SEQ ID NO:20, amino acids KVS, and SEQ ID NO: 13, respectively. In some embodiments, an isolated anti-ATP6V1B2 antibody disclosed herein comprises a light chain variable region having complementarity determining region 1 (L-CDR1), L-CDR2 and L-CDR3, wherein the L-CDR1, L-CDR2 and L-CDR3 comprise amino acid sequences of SEQ ID NO:29, amino acids KVS, and SEQ ID NO: 31, respectively. In certain embodiments of an isolated anti-ATP6V1B2 antibody, the amino acid sequence of the light chain variable region comprises one or more humanized framework (FR) sequences.

In some embodiments, an isolated anti-ATP6V1B2 antibody disclosed herein comprises a heavy chain variable region having complementarity determining region 1 (H-CDR1), H-CDR2 and H-CDR3, wherein the H-CDR1, H-CDR2 and H-CDR3 comprise amino acid sequences of SEQ ID NOs: 2, 4 and 6, respectively, and a light chain variable region having complementarity determining region 1 (L-CDR1), L-CDR2 and L-CDR3, wherein the L-CDR1, L-CDR2 and L-CDR3 comprise amino acid sequences of SEQ ID NO:9, amino acids KVS, and SEQ ID NO: 13, respectively. In some embodiments, an isolated anti-ATP6V1B2 antibody disclosed herein comprises a heavy chain variable region having complementarity determining region 1 (H-CDR1), H-CDR2 and H-CDR3, wherein the H-CDR1, H-CDR2 and H-CDR3 comprise amino acid sequences of SEQ ID NOs: 2, 4, and 6, respectively; and a light chain variable region having complementarity determining region 1 (L-CDR1), L-CDR2 and L-CDR3, wherein the L-CDR1, L-CDR2 and L-CDR3 comprise amino acid sequences of SEQ ID NO: 20, amino acids KVS, and SEQ ID NO: 13, respectively. In some embodiments, an isolated anti-ATP6V1B2 antibody disclosed herein comprises a heavy chain variable region having complementarity determining region 1 (H-CDR1), H-CDR2 and H-CDR3, wherein the H-CDR1, H-CDR2 and H-CDR3 comprise amino acid sequences of SEQ ID NOs: 24, 26, and 6, respectively; and a light chain variable region having complementarity determining region 1 (L-CDR1), L-CDR2 and L-CDR3, wherein the L-CDR1, L-CDR2 and L-CDR3 comprise amino acid sequences of SEQ ID NO: 29, amino acids KVS, SEQ ID NO: 31, respectively. In one embodiment, the present disclosure also encompasses a composition comprising the above-mentioned antibody and a pharmaceutically acceptable carrier.

In certain embodiments of an isolated anti-ATP6V1B2 antibody, the amino acid sequence of the heavy chain variable region comprises one or more humanized framework (FR) sequences and the amino acid sequence of the light chain variable region comprises one or more humanized framework (FR) sequences.

In certain embodiments of an isolated anti-ATP6V1B2 antibody comprising the H-CDR1, H-CDR2 and H-CDR3, and the L-CDR1, L-CDR2 and L-CDR3 disclosed herein, the antibody is an IgG, a Fv, a scFv, a Fab, or a F(ab′)2. The IgG can be of the subclass of IgG1, IgG2, IgG3, or IgG4. In one embodiment, the present disclosure also encompasses a composition comprising the above-mentioned antibody and a pharmaceutically acceptable carrier.

In certain embodiments, the present disclosure provides polypeptides comprising the VH and VL domains disclosed herein which could be dimerized under suitable conditions. For example, the VH and VL domains may be combined in a suitable buffer and dimerized through appropriate interactions such as hydrophobic interactions. In another embodiment, the VH and VL domains may be combined in a suitable buffer containing an enzyme and/or a cofactor which can promote dimerization of the VH and VL domains. In another embodiment, the VH and VL domains may be combined in a suitable vehicle that allows them to react with each other in the presence of a suitable reagent and/or catalyst.

In certain embodiments, the VH and VL domains disclosed herein may be contained within longer polypeptide sequences, which may include for example but not limited to, constant regions, hinge regions, linker regions, Fc regions, or disulfide binding regions, or any combination thereof. A constant domain is an immunoglobulin fold unit of the constant part of an immunoglobulin molecule, also referred to as a domain of the constant region (e.g., CH1, CH2, CH3, CH4, Ck, Cl).

In some embodiments, the present disclosure provides an anti-ATP6V1B2 antibody, wherein the antibody comprises a heavy chain variable region having the sequence of one of SEQ ID NOs: 15, 21 or 32. In certain embodiments, the antibody can be an IgG, a Fv, a scFv, a Fab, or a F(ab′)2. The IgG can be of the subclass of IgG1, IgG2, IgG3, or IgG4. In one embodiment, the present disclosure also encompasses a composition comprising the above-mentioned antibody and a pharmaceutically acceptable carrier.

In some embodiments, the present disclosure provides an anti-ATP6V1B2 antibody, wherein the antibody comprises a light chain variable region having the sequence of one of SEQ ID NOs: 16, 22 or 33. In certain embodiments, the antibody can be an IgG, a Fv, a scFv, a Fab, or a F(ab′)2. The IgG can be of the subclass of IgG1, IgG2, IgG3, or IgG4. In one embodiment, the present disclosure also encompasses a composition comprising the above-mentioned antibody and a pharmaceutically acceptable carrier.

In certain embodiments, the present disclosure provides an anti-ATP6V1B2 antibody, wherein the antibody comprises a heavy chain variable region and a light chain variable region having the sequences of one of: SEQ ID NOs: 15 and 16; SEQ ID NOs: 21 and 22; or SEQ ID NOs: 32 and 33. In certain embodiments, the antibody can be an IgG, a Fv, a scFv, a Fab, or a F(ab′)2. The IgG can be of the subclass of IgG1, IgG2, IgG3, or IgG4. In one embodiment, the present disclosure also encompasses a composition comprising the above-mentioned antibody and a pharmaceutically acceptable carrier.

In some embodiments, an anti-ATP6V1B2 antibody disclosed herein binds a human ATP6V1B2. In some embodiments, an anti-ATP6V1B2 antibody disclosed herein binds a non-human mammalian ATP6V1B2.

In some embodiments, an anti-ATP6V1B2 antibody disclosed herein binds human ATP6V1B2 and ATP6V1B1. In some embodiments, an anti-ATP6V1B2 antibody disclosed herein binds non-human mammalian ATP6V1B2 and ATP6V1B1.

In some embodiments, an anti-ATP6V1B2 antibody disclosed herein binds an ATP6V1B2 antigen on the cell surface. In some embodiments, an anti-ATP6V1B2 antibody disclosed herein binds both ATP6V1B2 and ATP6V1B1 antigens on the cell surface. In some embodiments, a non-senescent, growing cell does not express ATP6V1B2 on the cells surface. In some embodiments, an anti-ATP6V1B2 antibody disclosed herein does not bind to the cell surface of a growing (proliferative), or non-senescent cell. In some embodiments, an anti-ATP6V1B2 antibody disclosed herein binds an ATP6V1B2 antigen on the cell surface of a senescent cell. In some embodiments, an anti-ATP6V1B2 antibody disclosed herein binds a ATP6V1B2 antigen and an ATP6V1B1 antigen on the cell surface of a senescent cell.

In some embodiments, an anti-ATP6V1B2 antibody disclosed herein binds a ATP6V1B2 antigen on the cell surface of a senescent cell present within diseased tissue. In some embodiments, an anti-ATP6V1B2 antibody disclosed herein binds an ATP6V1B2 antigen and an ATP6V1B1 antigen on the cell surface of a senescent cell present within diseased tissue. In some embodiments, an anti-ATP6V1B2 antibody disclosed herein binds an ATP6V1B2 antigen on the cell surface of an age-related disease associated senescent cell. In some embodiments, an anti-ATP6V1B2 antibody disclosed herein binds an ATP6V1B2 antigen and an ATP6V1B1 antigen on the cell surface of an age-related disease associated senescent cell. In some embodiments, the cell tissue is fibrotic tissue. In some embodiments, the cell tissue is lung (pulmonary) tissue. In some embodiments, the cell tissue is hepatic tissue. In some embodiments, an anti-ATP6V1B2 antibody disclosed herein binds an ATP6V1B2 antigen on the cell surface of a senescent cell comprised in idiopathic pulmonary fibrotic (IPF) tissue. In some embodiments, the cell tissue comprises senescent cancer or tumor cells.

### Optimization of Anti-ATP6V1B2 Antibodies

In certain embodiments, the anti-ATP6V1B2 antibody is optimized. In certain embodiments, the anti-ATP6V1B2 antibody further comprises a heavy chain fragment crystallizable region (Fc region), wherein said Fc region comprises at least one amino acid residue substitution comprising S239D, 1332E, A330L, G236A, H268F, S324T, S267E, or any combination thereof; wherein fucosylation of the Fc region is reduced in comparison to a Fc region of an anti-ATP6V1B2 antibody produced in the presence of fucose; or both. In certain embodiments, the anti-ATP6V1B2 antibody further comprises a Fc region comprising at least one amino acid residue substitution combinations comprising S239D/1332E, A330L/S239D/1332E, G236A/1332E, H268F/S324T, S267E/H268F/S324T, or any combination thereof. In some embodiments, the anti-ATP6V1B2 antibody further comprises a Fc region comprising two amino acid residue substitution combinations comprising S239D/1332E, G236A/1332E, or H268F/S324T. In some embodiments, the anti-ATP6V1B2 antibody further comprises a Fc region comprising two amino acid residue substitutions comprising S239D/1332E. In some embodiments, the anti-ATP6V1B2 antibody further comprises a Fc region comprising two amino acid residue substitutions comprising G236A/1332E. In some embodiments, the anti-ATP6V1B2 antibody further comprises a Fc region comprising two amino acid residue substitutions comprising H268F/S324T. In some embodiments, the anti-ATP6V1B2 antibody further comprises a Fc region comprising three amino acid residue substitutions comprising A330L/S239D/1332E or S267E/H268F/S324T. In some embodiments, the anti-ATP6V1B2 antibody further comprises a Fc region comprising three amino acid residue substitutions comprising A330L/S239D/1332E. In some embodiments, the anti-ATP6V1B2 antibody further comprises a Fc region comprising three amino acid residue substitutions comprising S267E/H268F/S324T.

In certain embodiments, the Fc region is afucosylated. In certain embodiments, the antibodies disclosed herein are produced in the absence of fucose.

In certain embodiments, the anti-ATP6V1B2 antibody comprises at least two substitution mutations and the Fc region is afucosylated, wherein said substitution mutations comprise any of S239D/1332E, G236A/1332E, or H268F/S324T and the Fc region is afucosylated. In certain embodiments, the anti-ATP6V1B2 antibody comprises at least three substitution mutations comprising any of A330L/S239D/1332E or S267E/H268F/S324T and the Fc region is afucosylated.

In some embodiments, the anti-ATP6V1B2 antibody further comprises a Fc region comprising two amino acid residue substitution combinations comprising S239D/1332E, G236A/1332E, or H268F/S324T and the Fc region is afucosylated. In some embodiments, the anti-ATP6V1B2 antibody further comprises a Fc region comprising two amino acid residue substitutions comprising S239D/1332E and the Fc region is afucosylated. In some embodiments, the anti-ATP6V1B2 antibody further comprises a Fc region comprising two amino acid residue substitutions comprising G236A/1332E and the Fc region is afucosylated. In some embodiments, the anti-ATP6V1B2 antibody further comprises a Fc region comprising two amino acid residue substitutions comprising H268F/S324T and the Fc region is afucosylated. In some embodiments, the anti-ATP6V1B2 antibody further comprises a Fc region comprising three amino acid residue substitutions comprising A330L/S239D/1332E or S267E/H268F/S324T and the Fc region is afucosylated. In some embodiments, the anti-ATP6V1B2 antibody further comprises a Fc region comprising three amino acid residue substitutions comprising A330L/S239D/1332E and the Fc region is afucosylated. In some embodiments, the anti-ATP6V1B2 antibody further comprises a Fc region comprising three amino acid residue substitutions comprising S267E/H268F/S324T and the Fc region is afucosylated.

In certain embodiments, binding of the anti-ATP6V1B2 antibody comprising the at least one amino acid residue substitution to an ATP6V1B2 epitope moiety enhances antibody-dependent cell-mediated cytotoxicity (ADCC), complement dependent cytotoxicity (CDC), antibody-dependent cellular phagocytosis (ADCP), or any combination thereof in comparison with binding of a wild-type anti-ATP6V1B2 antibody to the ATP6V1B2 epitope moiety.

In certain embodiments, binding of the anti-ATP6V1B2 antibody to an ATP6V1B2 epitope moiety enhances antibody-dependent cell-mediated cytotoxicity (ADCC), complement dependent cytotoxicity (CDC), antibody-dependent cellular phagocytosis (ADCP), or any combination thereof in comparison with binding of an anti-ATP6V1B2 antibody produced in the presence of fucose to the ATP6V1B2 epitope moiety.

### Polynucleotides Encoding Anti-ATP6V1B2 Antibodies

In certain embodiments, the present disclosure provides isolated polynucleotides encoding a polypeptide comprising a variable heavy chain region (VH) of an anti-ATP6V1B2 antibody disclosed herein. In certain embodiments, the present disclosure provides isolated polynucleotides encoding a polypeptide comprising a variable light chain region (VH) of an anti-ATP6V1B2 antibody disclosed herein. In certain embodiments, the present disclosure provides isolated polynucleotides encoding a single polypeptide comprising the VH and VL domains of an anti-ATP6V1B2 antibody disclosed herein, which could be dimerized under suitable conditions. In certain embodiments, the present disclosure provides isolated polynucleotides encoding a polypeptide comprising the VH disclosed herein and a polypeptide comprising the VL domains of an anti-ATP6V1B2 antibody disclosed herein, which could be dimerized under suitable conditions.

In some embodiments, the present disclosure provides a polynucleotide encoding a polypeptide comprising an anti-ATP6V1B2 antibody heavy chain variable region having the amino acid sequence of one of SEQ ID NOs: 15, 21 or 32.

In some embodiments, the present disclosure provides a polynucleotide encoding a polypeptide comprising an anti-ATP6V1B2 antibody light chain variable region having the amino acid sequence of one of SEQ ID NOs: 16, 22 or 33.

In some embodiments, the present disclosure provides a polynucleotide encoding a polypeptide comprising an anti-ATP6V1B2 antibody heavy chain variable region (VH) and a light chain variable region (VL), wherein the VH and VL comprise the amino acid sequences of SEQ ID NO: 15 and 16, or SEQ ID NO:21 and 22, or SEQ ID NO: 32 and 33.

In certain embodiments, an isolated polynucleotide described herein is inserted into a vector. The term “vector” as used herein encompasses a vehicle into which a polynucleotide encoding a protein may be covalently inserted so as to bring about the expression of that protein and/or the cloning of the polynucleotide. The isolated polynucleotide may be inserted into a vector using any suitable methods known in the art, for example, without limitation, the vector may be digested using appropriate restriction enzymes and then may be ligated with the isolated polynucleotide having matching restriction ends. In some embodiments, a vector disclosed herein comprises a single polynucleotide sequence. In some embodiments, a vector disclosed herein comprises two polynucleotide sequences.

Examples of suitable vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40).

For expression of the polypeptide, the vector may be introduced into a host cell to allow expression of the polypeptide within the host cell. In some embodiments, a host cells comprises a CHO or a HEK293 tissue culture cell. The expression vectors may contain a variety of elements for controlling expression, including without limitation, promoter sequences, transcription initiation sequences, enhancer sequences, selectable markers, and signal sequences. These elements may be selected as appropriate by a person of ordinary skill in the art. For example, the promoter sequences may be selected to promote the transcription of the polynucleotide in the vector. Suitable promoter sequences include, without limitation, T7 promoter, T3 promoter, SP6 promoter, beta-actin promoter, EF1a promoter, CMV promoter, and SV40 promoter. Enhancer sequences may be selected to enhance the transcription of the polynucleotide. Selectable markers may be selected to allow selection of the host cells inserted with the vector from those not, for example, the selectable markers may be genes that confer antibiotic resistance. Signal sequences may be selected to allow the expressed polypeptide to be transported outside of the host cell.

In some embodiments, a host cell disclosed herein comprises a single vector. In some embodiments, a host cell disclosed herein comprises two vectors.

A vector may also include materials to aid in its entry into the cell, including but not limited to a viral particle, a liposome, or a protein coating.

For cloning of the polynucleotide, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

Suitable host cells can include, without limitation, prokaryotic cells, fungal cells, yeast cells, or higher eukaryotic cells such as mammalian cells.

### Compositions and Uses Thereof

In another aspect, described herein are methods of treating a disease in a subject, comprising the step of administering to the subject a composition comprising an effective amount of the anti-ATP6V1B2 antibody, wherein: (a) the anti-ATP6V1B2 antibody further comprises a heavy chain fragment crystallizable region (Fc region), wherein said Fc region comprises at least one amino acid residue substitution comprising S239D, 1332E, A330L, G236A, H268F, S324T, S267E, or any combination thereof; wherein fucosylation of the Fc region is reduced in comparison to a Fc region of an anti-ATP6V1B2 antibody produced in the presence of fucose; or both; (b) the anti-ATP6V1B2 antibody further comprises an ATP6V1B2 antibody-drug conjugate; or (c) both (a) and (b).

In some embodiments, described herein are pharmaceutical compositions comprising a ATP6V1B2 binding antibody, as described above in detail, which provides a therapeutic agent. In some embodiments, described herein are pharmaceutical compositions comprising an ATP6V1B2 binding antibody having therapeutic properties targeting senescent cells. In some embodiments, described herein are pharmaceutical compositions comprising an ATP6V1B2 binding antibody having therapeutic properties targeting senescent cells comprised in age-related diseases. A skilled artisan would appreciate that age-related diseases include diseases associated with cellular senescence. In some embodiments, age-related diseases comprise inflammatory diseases, fibrotic diseases, chronic fibrotic diseases, chronic fibrotic lung diseases, idiopathic pulmonary fibrosis (IPF), chronic obstructive pulmonary disease (COPD), cancer, nonalcoholic steatohepatitis (NASH), chronic liver disease, liver fibrosis, and chronic NASH. In some embodiments, described herein are pharmaceutical compositions comprising an ATP6V1B2 binding antibody having therapeutic properties targeting senescent cells in an age-related disease comprising inflammatory diseases, fibrotic diseases, chronic fibrotic diseases, chronic fibrotic lung diseases, idiopathic pulmonary fibrosis (IPF), chronic obstructive pulmonary disease (COPD), cancer, nonalcoholic steatohepatitis (NASH), chronic liver disease, liver fibrosis, and chronic NASH. In some embodiments, described herein are pharmaceutical compositions comprising an ATP6V1B2 binding antibody having therapeutic properties targeting senescent cells in pulmonary (lung) diseases. In some embodiments, described herein are pharmaceutical compositions comprising an ATP6V1B2 binding antibody having therapeutic properties targeting senescent cells in liver diseases. In some embodiments, described herein are pharmaceutical compositions comprising an ATP6V1B2 binding antibody having therapeutic properties targeting senescent cells in a fibrotic disease. In some embodiments, described herein are pharmaceutical compositions comprising an ATP6V1B2 binding antibody having therapeutic properties targeting senescent cells in an age-related cancer.

In some instances, therapeutic treatments of cancer result in the successful elimination (cytotoxicity) of a proportion of the targeted cancer cells, while a residual population of senescent cancer cells remain. Re-entry of these senescent cells into the cell cycle, leads to a devastating return of the cancer. In some embodiments, described herein are pharmaceutical compositions comprising an ATP6V1B2 binding antibody having therapeutic properties targeting senescent cancer cells.

In some embodiments, pharmaceutical compositions comprising a ATP6V1B2 antibody target senescent cells comprised in pulmonary tissue. In some embodiments, pharmaceutical compositions comprising a ATP6V1B2 antibody target senescent cells comprised in lung tissue. In some embodiments, pharmaceutical compositions comprising a ATP6V1B2 antibody target senescent cells comprised in liver tissue. In some embodiments, pharmaceutical compositions comprising a ATP6V1B2 antibody target senescent cells comprised in fibrotic tissue. In some embodiments, pharmaceutical compositions comprising a ATP6V1B2 antibody target senescent cancer cells. In some embodiments, pharmaceutical compositions comprising a ATP6V1B2 antibody target senescent cancer cells comprised in a tumor.

In some embodiments, pharmaceutical compositions comprising a ATP6V1B2 antibody target senescent cells comprised in diseased tissue. In some embodiments, pharmaceutical compositions comprising an ATP6V1B2 antibody target senescent cells comprised in diseased lung tissue. In some embodiments, pharmaceutical compositions comprising an ATP6V1B2 antibody target senescent cells comprised in diseased pulmonary tissue. In some embodiments, pharmaceutical compositions comprising an ATP6V1B2 antibody target senescent cells comprised in diseased liver tissue. In some embodiments, pharmaceutical compositions comprising an ATP6V1B2 antibody target senescent cells comprised in diseased tissue, wherein the disease comprises an age-related disease. In some embodiments, pharmaceutical compositions comprising an ATP6V1B2 antibody target senescent cells comprised in diseased tissue, wherein the disease comprises a cancer or tumor. In some embodiments, pharmaceutical compositions comprising an ATP6V1B2 antibody target senescent cells comprised in diseased tissue, wherein the disease comprises an inflammatory disease. In some embodiments, pharmaceutical compositions comprising an ATP6V1B2 antibody target senescent cells comprised in diseased tissue, wherein the disease comprises a fibrotic disease.

In some embodiments, pharmaceutical compositions comprising an ATP6V1B2 antibody target senescent cells relevant in age-related inflammatory diseases. In some embodiments, pharmaceutical compositions comprising an ATP6V1B2 antibody target senescent cells relevant in age-related fibrotic diseases.

In some embodiments, pharmaceutical compositions comprising an ATP6V1B2 antibody target senescent fibroblast cells relevant in an age-related disease. In some embodiments, pharmaceutical compositions comprising an ATP6V1B2 antibody target senescent epithelial cells relevant in an age-related disease. In some embodiments, pharmaceutical compositions comprising an ATP6V1B2 antibody target senescent cancer cells relevant in an age-related disease. In some embodiments, pharmaceutical compositions comprising an ATP6V1B2 antibody target senescent endothelial cells relevant in an age-related disease. In some embodiments, pharmaceutical compositions comprising an ATP6V1B2 antibody target senescent immune cells relevant in an age-related disease. In some embodiments, pharmaceutical compositions comprising an ATP6V1B2 antibody target senescent cancer cells. In some embodiments, pharmaceutical compositions comprising an ATP6V1B2 antibody target senescent cancer cells in a tumor.

In some embodiments, pharmaceutical compositions comprising an ATP6V1B2 antibody target senescent fibroblast cells relevant in a chronic fibrotic disease. In some embodiments, pharmaceutical compositions comprising an ATP6V1B2 antibody target senescent fibroblast cells relevant in a fibrotic lung disease. In some embodiments, pharmaceutical compositions comprising an ATP6V1B2 antibody target senescent fibroblast cells relevant in a chronic fibrotic lung disease. In some embodiments, pharmaceutical compositions comprising an ATP6V1B2 antibody target senescent fibroblast cells relevant in idiopathic pulmonary fibrosis (IPF). In some embodiments, pharmaceutical compositions comprising an ATP6V1B2 antibody target senescent fibroblast cells relevant in chronic obstructive pulmonary disease (COPD).

In some embodiments, pharmaceutical compositions comprising an ATP6V1B2 antibody target senescent epithelial cells relevant in a chronic fibrotic disease. In some embodiments, pharmaceutical compositions comprising an ATP6V1B2 antibody target senescent epithelial cells relevant in a fibrotic lung disease. In some embodiments, pharmaceutical compositions comprising an ATP6V1B2 antibody target senescent epithelial cells relevant in a chronic fibrotic lung disease. In some embodiments, pharmaceutical compositions comprising an ATP6V1B2 antibody target senescent epithelial cells relevant in idiopathic pulmonary fibrosis (IPF). In some embodiments, pharmaceutical compositions comprising an ATP6V1B2 antibody target senescent epithelial cells relevant in chronic obstructive pulmonary disease (COPD).

In some embodiments, pharmaceutical compositions comprising an ATP6V1B2 antibody target senescent epithelial cells relevant in an inflammatory disease. In some embodiments, pharmaceutical compositions comprising an ATP6V1B2 antibody target senescent epithelial cells relevant in NASH. In some embodiments, pharmaceutical compositions comprising an ATP6V1B2 antibody target senescent epithelial cells relevant in chronic NASH.

In some embodiments, pharmaceutical compositions comprising an ATP6V1B2 antibody target senescent immune cells relevant in an inflammatory disease. In some embodiments, pharmaceutical compositions comprising an ATP6V1B2 antibody target senescent immune cells relevant in NASH. In some embodiments, pharmaceutical compositions comprising an ATP6V1B2 antibody target senescent immune cells relevant in chronic NASH.

In some embodiments, pharmaceutical compositions comprising an ATP6V1B2 antibody target senescent endothelial cells relevant in an inflammatory disease. In some embodiments, pharmaceutical compositions comprising an ATP6V1B2 antibody target senescent endothelial cells relevant in NASH. In some embodiments, pharmaceutical compositions comprising an ATP6V1B2 antibody target senescent endothelial cells relevant in chronic NASH.

In some embodiments, pharmaceutical compositions comprising an ATP6V1B2 antibody target senescent cancer cells, including but not limited to endothelial, epithelial, or immune cancer cells.

In some embodiments, pharmaceutical compositions comprising an ATP6V1B2 antibody target senescent fibroblast cells relevant in an inflammatory disease. In some embodiments, pharmaceutical compositions comprising an ATP6V1B2 antibody target senescent fibroblast cells relevant in NASH. In some embodiments, pharmaceutical compositions comprising an ATP6V1B2 antibody target senescent fibroblast cells relevant in chronic NASH.

In some embodiments, use of an ATP6V1B2 antibody or a composition comprising an ATP6V1B2 antibody induces antibody-dependent cell-mediated cytotoxicity (ADCC) in senescent cells. A skilled artisan would appreciate that ADCC may also be referred to as antibody-dependent cellular cytotoxicity. In some embodiments, use of an ATP6V1B2 antibody or a composition comprising an ATP6V1B2 antibody induces antibody-dependent cellular cytotoxicity (ADCC) wherein a target cell is lysed, or other types of cytotoxicity including Complement Dependent Cytotoxicity (CDC).

In some embodiments, use of an ATP6V1B2 antibody or a composition comprising an ATP6V1B2 antibody induces antibody-dependent cell-mediated cytotoxicity (ADCC) in an epithelial senescent cell. In some embodiments, use of an ATP6V1B2 antibody or a composition comprising an ATP6V1B2 antibody induces ADCC wherein a target senescent epithelial cell is lysed.

In some embodiments, use of an ATP6V1B2 antibody or a composition comprising an ATP6V1B2 antibody induces antibody-dependent cell-mediated cytotoxicity (ADCC) in a fibroblast senescent cell. In some embodiments, use of an ATP6V1B2 antibody or a composition comprising an ATP6V1B2 antibody induces ADCC wherein a target senescent fibroblast cell is lysed.

In some embodiments, use of an ATP6V1B2 antibody or a composition comprising an ATP6V1B2 antibody induces antibody-dependent cell-mediated cytotoxicity (ADCC) in a senescent lung cell, wherein the disease is a lung disease. In some embodiments, use of an ATP6V1B2 antibody or a composition comprising an ATP6V1B2 antibody induces antibody-dependent cell-mediated cytotoxicity (ADCC) in a senescent epithelial lung cell, wherein the disease is lung disease. In some embodiments, use of an ATP6V1B2 antibody or a composition comprising an ATP6V1B2 antibody induces antibody-dependent cell-mediated cytotoxicity (ADCC) towards senescent lung cells in diseased lung tissue. In some embodiments, use of an ATP6V1B2 antibody or a composition comprising an ATP6V1B2 antibody induces antibody-dependent cell-mediated cytotoxicity (ADCC) towards senescent lung cells during treatment of a lung disease.

In some embodiments, use of an ATP6V1B2 antibody or a composition comprising an ATP6V1B2 antibody induces antibody-dependent cell-mediated cytotoxicity (ADCC) in a senescent cell, wherein the disease is an age-related disease. In some embodiments, the senescent cell identified is comprised in diseased tissue. In some embodiments, use of an ATP6V1B2 antibody or a composition comprising an ATP6V1B2 antibody induces antibody-dependent cell-mediated cytotoxicity (ADCC) in a senescent cell, wherein the disease is an inflammatory disease. In some embodiments, use of an ATP6V1B2 antibody or a composition comprising an ATP6V1B2 antibody induces antibody-dependent cell-mediated cytotoxicity (ADCC) in a senescent cell, wherein the disease is a fibrotic disease.

In some embodiments, use of an ATP6V1B2 antibody or a composition comprising an ATP6V1B2 antibody induces antibody-dependent cell-mediated cytotoxicity (ADCC) in a senescent cell, wherein the disease is an inflammatory disease including but not limited to NASH or chronic NASH. In some embodiments, use of an ATP6V1B2 antibody or a composition comprising an ATP6V1B2 antibody induces antibody-dependent cell-mediated cytotoxicity (ADCC) in a senescent cell, wherein the disease is a fibrotic disease including but not limited to chronic fibrotic diseases, fibrotic lung diseases, chronic fibrotic lung diseases, idiopathic pulmonary fibrosis (IPF), and chronic obstructive pulmonary disease (COPD).

In some embodiments, use of an ATP6V1B2 antibody or a composition comprising an ATP6V1B2 antibody induces antibody-dependent cell-mediated cytotoxicity (ADCC) in a senescent cancer cell.

In some embodiments, use of an ATP6V1B2 antibody comprises use of ATP6V1B2 antibody-drug conjugate (ADC). In some embodiments, use of an ATP6V1B2 antibody conjugate comprises an ATP6V1B2 antibody conjugated to a pharmaceutical agent, a nucleic acid, a protein, a peptide, a polypeptide or polynucleotide vector, an imaging agent, a biomarker, a medicament, a chemotherapeutic agent, a cytotoxic agent, a toxin, or a radioactive isotope. In some embodiments, the drug comprises a chemotherapeutic or cytotoxic agent. In some embodiments, use of an ATP6V1B2 antibody composition comprises use of an ATP6V1B2 ADC composition.

In some embodiments, described herein are pharmaceutical compositions comprising polynucleotides that encode an ATP6V1B2 binding antibody or portions thereof, as described above in detail, which provides a therapeutic agent. In some embodiments, described herein are pharmaceutical compositions comprising a polynucleotide that encodes an ATP6V1B2 binding antibody having therapeutic properties targeting senescent cells. In some embodiments, described herein are pharmaceutical compositions comprising multiple polynucleotides that together encode an ATP6V1B2 binding antibody having therapeutic properties targeting senescent cells. In some embodiments, described herein are pharmaceutical compositions comprising two polynucleotides that together encode an ATP6V1B2 binding antibody having therapeutic properties targeting senescent cells. Polynucleotides encoding VH, VL, or VH and VL have been described in detail above.

In some embodiments, a pharmaceutical composition comprises an ATP6V1B2 binding antibody comprising a VH, a VL, or a VH and a VL, and a pharmaceutically acceptable carrier. The amino acid sequences of VH and VL domains, and pairs thereof, have been described in detail above.

In some embodiments, a pharmaceutical composition comprising a ATP6V1B2 binding antibody comprises any ATP6V1B2 antibody described herein comprising a VH, a VL, or a VH and a VL.

A skilled artisan would recognize that in some embodiments, the terms “ATP6V1B2 binding antibody” or “isolated anti-ATP6V1B2 antibody” or “anti-ATP6V1B2 antibody” may be used interchangeably with the term “drug” or “agent” having all the same meanings and qualities. In some embodiments, a drug comprising an ATP6V1B2 binding antibody comprises a pharmaceutical composition.

The anti-ATP6V1B2 antibodies disclosed herein can in certain embodiments, be administered to a subject (e.g., a human or an animal) alone, or in combination with a carrier, i.e., a pharmaceutically acceptable carrier. By pharmaceutically acceptable is meant a material that is not biologically or otherwise undesirable, i.e., the material can be administered to a subject without causing any undesirable biological effects or interacting in a deleterious manner with any of the other components of the pharmaceutical composition in which it is contained. As would be well-known to one of ordinary skill in the art, the carrier is selected to minimize any degradation of the polypeptides disclosed herein and to minimize any adverse side effects in the subject. The pharmaceutical compositions may be prepared by methodology well known in the pharmaceutical art.

The above pharmaceutical compositions comprising anti-ATP6V1B2 antibody disclosed herein can be administered (e.g., to a mammal, a cell, or a tissue) in any suitable manner depending on whether local or systemic treatment is desired. For example, the composition can be administered by local or intravenous injection. In some embodiments, administration comprises intravenous (iv) injection.

If the composition is to be administered parenterally, the administration is generally by injection. Injectables can be prepared in conventional forms, either as liquid solutions or suspensions, solid forms suitable for suspension in liquid prior to injection, or as emulsions.

Additionally, parental administration can involve preparation of a slow-release or sustained-release system so as to maintain a constant dosage.

### Methods of Use

In one embodiment, the present disclosure provides a method of producing a heavy chain variable region of an anti-ATP6V1B2 antibody, the method comprises the step of culturing host cells under conditions conducive to expressing a vector comprising a polynucleotide encoding the heavy chain variable region, thereby producing the heavy chain variable region of the anti-ATP6V1B2 antibody.

In one embodiment, the present disclosure provides a method of producing a light chain variable region of an anti-ATP6V1B2 antibody, the method comprises the step of culturing host cells under conditions conducive to expressing a vector encoding a polynucleotide encoding the light chain variable region, thereby producing the light chain variable region of the anti-ATP6V1B2 antibody.

In one embodiment, the present disclosure provides a method of producing a heavy chain variable region and a light chain variable region of an anti-ATP6V1B2 antibody, the method comprises the step of culturing at least one host cell under conditions conducive to expressing a vector comprising a polynucleotide encoding the heavy chain variable region and a vector encoding the light chain variable region, wherein the vector may be the same or a different vector, thereby producing a polypeptide comprising the heavy chain variable region and a polypeptide comprising the light chain variable region of the anti-ATP6V1B2 antibody. In one embodiment, the present disclosure provides a method of producing a heavy chain variable region and a light chain variable region of an anti-ATP6V1B2 antibody in a single polypeptide, the method comprises the step of culturing host cells under conditions conducive to expressing a vector comprising a polynucleotide encoding the heavy chain variable region and the light chain variable region, thereby producing a polypeptide comprising the heavy chain variable region and the light chain variable region of the anti-ATP6V1B2 antibody. In some embodiments, the polypeptide comprises for example but not limited to, an scFv.

The anti-ATP6V1B2 antibody disclosed herein may be used in therapeutic methods. In some embodiments, described herein are methods of treating a disease in a subject, comprising the step of administering to the subject a composition comprising an effective amount of the anti-ATP6V1B2 antibody, wherein: (a) the anti-ATP6V1B2 antibody further comprises a heavy chain fragment crystallizable region (Fc region), wherein said Fc region comprises at least one amino acid residue substitution comprising S239D, 1332E, A330L, G236A, H268F, S324T, S267E, or any combination thereof; wherein fucosylation of the Fc region is reduced in comparison to a Fc region of an anti-ATP6V1B2 antibody produced in the presence of fucose; or both; (b) the anti-ATP6V1B2 antibody further comprises an ATP6V1B2 antibody-drug conjugate; or (c) both (a) and (b).

In some embodiments, a method of treating a disease in a subject, comprises the step of administering to the subject a composition comprising an effective amount of an anti-ATP6V1B2 antibody, the anti-ATP6V1B2 antibody further comprising a Fc region comprising two amino acid residue substitution combinations comprising S239D/1332E, G236A/1332E, or H268F/S324T. In some embodiments, a method of treating a disease in a subject, comprises the step of administering to the subject a composition comprising an effective amount of an anti-ATP6V1B2 antibody, the anti-ATP6V1B2 antibody further comprising a Fc region comprising two amino acid residue substitutions comprising S239D/1332E. In some embodiments, a method of treating a disease in a subject, comprises the step of administering to the subject a composition comprising an effective amount of an anti-ATP6V1B2 antibody, the anti-ATP6V1B2 antibody further comprising a Fc region comprising two amino acid residue substitutions comprising G236A/1332E. In some embodiments, a method of treating a disease in a subject, comprises the step of administering to the subject a composition comprising an effective amount of an anti-ATP6V1B2 antibody, the anti-ATP6V1B2 antibody further comprising a Fc region comprising two amino acid residue substitutions comprising H268F/S324T. In some embodiments, a method of treating a disease in a subject, comprises the step of administering to the subject a composition comprising an effective amount of an anti-ATP6V1B2 antibody, the anti-ATP6V1B2 antibody further comprising a Fc region comprising three amino acid residue substitutions comprising A330L/S239D/1332E or S267E/H268F/S324T In some embodiments, a method of treating a disease in a subject, comprises the step of administering to the subject a composition comprising an effective amount of an anti-ATP6V1B2 antibody, the anti-ATP6V1B2 antibody further comprising a Fc region comprising three amino acid residue substitutions comprising A330L/S239D/1332E. In some embodiments, a method of treating a disease in a subject, comprises the step of administering to the subject a composition comprising an effective amount of an anti-ATP6V1B2 antibody, the anti-ATP6V1B2 antibody further comprising a Fc region comprising three amino acid residue substitutions comprising S267E/H268F/S324T.

In some embodiments, a method of treating a disease in a subject, comprises the step of administering to the subject a composition comprising an effective amount of an anti-ATP6V1B2 antibody, the anti-ATP6V1B2 antibody further comprising a Fc region comprising substitution mutation and as well, the Fc region is afucosylated. In certain embodiments, for method of treating a disease, the antibodies administered are produced in the absence of fucose.

In some embodiments, a method of treating a disease in a subject, comprises the step of administering to the subject a composition comprising an effective amount of an anti-ATP6V1B2 antibody, the anti-ATP6V1B2 antibody further comprising a Fc region comprising at least two substitution mutations and the Fc region is afucosylated, wherein said substitution mutations comprise any of S239D/1332E, G236A/1332E, or H268F/S324T. In some embodiments, a method of treating a disease in a subject, comprises the step of administering to the subject a composition comprising an effective amount of an anti-ATP6V1B2 antibody, the anti-ATP6V1B2 antibody further comprising a Fc region comprising at least three substitution mutations comprising any of A330L/S239D/1332E or S267E/H268F/S324T and the Fc region is afucosylated.

In some embodiments, a method of treating a disease in a subject, comprises the step of administering to the subject a composition comprising an effective amount of an anti-ATP6V1B2 antibody comprising a Fc region comprising two amino acid residue substitution combinations comprising S239D/1332E, G236A/1332E, or H268F/S324T and the Fc region is afucosylated. In some embodiments, a method of treating a disease in a subject, comprises the step of administering to the subject a composition comprising an effective amount of an anti-ATP6V1B2 antibody comprising a Fc region comprising two amino acid residue substitutions comprising S239D/1332E and the Fc region is afucosylated. In some embodiments, a method of treating a disease in a subject, comprises the step of administering to the subject a composition comprising an effective amount of an anti-ATP6V1B2 antibody comprising a Fc region comprising two amino acid residue substitutions comprising G236A/1332E and the Fc region is afucosylated. In some embodiments, a method of treating a disease in a subject, comprises the step of administering to the subject a composition comprising an effective amount of an anti-ATP6V1B2 antibody comprising a Fc region comprising two amino acid residue substitutions comprising H268F/S324T and the Fc region is afucosylated. In some embodiments, a method of treating a disease in a subject, comprises the step of administering to the subject a composition comprising an effective amount of an anti-ATP6V1B2 antibody comprising a Fc region comprising three amino acid residue substitutions comprising A330L/S239D/1332E or S267E/H268F/S324T and the Fc region is afucosylated. In some embodiments, a method of treating a disease in a subject, comprises the step of administering to the subject a composition comprising an effective amount of an anti-ATP6V1B2 antibody comprising a Fc region comprising three amino acid residue substitutions comprising A330L/S239D/1332E and the Fc region is afucosylated. In some embodiments, a method of treating a disease in a subject, comprises the step of administering to the subject a composition comprising an effective amount of an anti-ATP6V1B2 antibody comprising a Fc region comprising three amino acid residue substitutions comprising S267E/H268F/S324T and the Fc region is afucosylated.

In some embodiments, the anti-ATP6V1B2 antibody of the present disclosure can be used to target senescent cells. A skilled artisan would appreciate that “senescent cells” encompasses cells that exhibit cell cycle arrest, generally during the G1 transition of the cell cycle or in few cases in G2, elicited by replicative exhaustion due to telomere attrition or in response to stresses such as DNA damage, chemotherapeutic drugs, or aberrant expression of oncogenes. According to some embodiments, senescent cells are characterized by at least one or more of the following characteristics: (1) activation of the p53/p21CIP1 and/or pRb/p16INK4A tumor suppressor pathways; (2) cells whose proliferation is irreversibly arrested; (3) shortening of telomere size; (4) expression of senescent-associated beta-galactosidase activity; (5) specific chromatin modification; (6) specific secretome; (7) increase in reactive oxygen species and altered overall mitochondrial activity.

Senescent cells may be identified using technology known in the art, for example but not limited to the following assays: irreversible cell cycle arrest of may be assessed by FACS or BrdU incorporation assay, and shortening of telomere size may be characterized by evaluating the mean terminal restriction fragment (TRF) length for example by Southern blot analysis. Other methods of ascertaining whether a cell is senescent are described in U.S. Pat. No. 9,476,031 and Biran et al, 2017 Aging Cell 16:661-7. Doi: 10.1111/acel. 12592) the contents of which are incorporated herein by reference.

In some embodiments, a method of use of an anti-ATP6V1B2 antibody disclosed herein comprises treating a disease or condition related to cellular senescence. In some embodiments, a disease or condition related to cellular senescence comprises age-related diseases. In certain embodiments, age-related diseases that could be treating using a method of use disclosed herein comprise inflammatory diseases, fibrotic diseases, chronic fibrotic diseases, chronic fibrotic lung diseases, idiopathic pulmonary fibrosis (IPF), chronic obstructive pulmonary disease (COPD), cancer, nonalcoholic steatohepatitis (NASH), chronic liver disease, liver fibrosis, and chronic NASH.

In some embodiments, a method of use of an anti-ATP6V1B2 antibody disclosed herein comprises treating a disease or condition related to cancer.

In some embodiments, the present disclosure provides a method of targeting a senescent cell in a subject, comprising the step of preparing a composition comprising an anti-ATP6V1B2 antibody disclosed herein; and administering the composition to the subject, thereby targeting the senescent cell in the subject. In some embodiments, the subject can be an animal or a human. In some embodiments, the present disclosure provides a method of targeting a senescent cell in a subject in need wherein said subject suffers from an age-related disease, comprising the step of preparing a composition comprising the anti-ATP6V1B2 antibody disclosed herein; and administering the composition to the subject suffering from an age-related disease, thereby targeting the senescent cell in the subject in need.

In some embodiments, the present disclosure provides a method of treating a disease associated with cellular senescence in a subject, comprising the step of preparing a composition comprising an anti-ATP6V1B2 antibody disclosed herein; and administering the composition to the subject, thereby treating the disease or condition associated with cellular senescence in the subject.

In some embodiments of methods of use to treat a subject in need disclosed herein, the anti-ATP6V1B2 antibodies of the present disclosure can be used to target senescent cells in a subject suffering from an age-related disease. In some embodiments of methods of use to treat a subject in need disclosed herein, the anti-ATP6V1B2 antibodies of the present disclosure can be used to target senescent cancer cells in a subject suffering from a cancer or a tumor.

In some embodiments, method of use of the compositions comprising an anti-ATP6V1B2 antibody described herein in comprise treating a subject suffering from an age-related disease including fibrotic or inflammatory diseases of skin, liver, lung, pancreas, prostate, articular cartilage, and atherosclerotic plaques. In some embodiments, method of use of the compositions comprising an anti-ATP6V1B2 antibody described herein in comprise treating a subject suffering from accumulation of senescent cells in normal tissues, especially skin that occurs with tissue aging. In some embodiments, method of use of the compositions comprising an anti-ATP6V1B2 antibody described herein in comprise treating a subject suffering from accumulation of senescent cells in normal tissues, especially lung tissue that occurs with tissue aging. In some embodiments, method of use of the compositions comprising an anti-ATP6V1B2 antibody described herein in comprise treating a subject suffering from accumulation of senescent cells in normal tissues, especially liver tissue that occurs with tissue aging.

The exact amount of the present anti-ATP6V1B2 antibodies or compositions thereof required to elicit the desired effects will vary from subject to subject, depending on the species, age, gender, weight, and general condition of the subject, the particular polypeptides, the route of administration, and whether other drugs are included in the regimen. Thus, it is not possible to specify an exact amount for every composition. However, an appropriate amount can be determined by one of ordinary skill in the art using routine experimentation. Dosages can vary, and the anti-ATP6V1B2 antibodies can be administered in one or more (e.g., two or more, three or more, four or more, or five or more) doses daily, for one or more days. Guidance in selecting appropriate doses for antibodies can be readily found in the literature.

In some embodiments, a method of treating an age-related disease reduces the severity and or extent of the age-related disease, compared with a subject not administered an anti-ATP6V1B2 antibody or a pharmaceutical composition thereof. In some embodiments, a method of treating an age-related disease reduces the duration of the disease in a subject compared with a subject not administered an anti-ATP6V1B2 antibody or a pharmaceutical composition thereof.

A skilled artisan would appreciate that the term “treating” and grammatical forms thereof, may in some embodiments encompass both therapeutic treatment and prophylactic or preventative measures with respect to diseases and conditions associated with cellular senescence or a tumor or cancer as described herein. With respect to treating a tumor or cancer, the object may be to prevent or lessen the targeted tumor or cancer as described herein. Thus, in some embodiments of methods disclosed herein, treating may include directly affecting or curing, suppressing, inhibiting, preventing, reducing the severity of, delaying the onset of, reducing symptoms associated with the disease, disorder or condition, or a combination thereof; for example, when said disease or disorder comprises a senescence related disease or condition. Thus, in some embodiments, “treating” encompasses preventing, delaying progression, inhibiting the growth of, delaying disease progression, reducing tumor load, reducing the incidence of, expediting remission, inducing remission, augmenting remission, speeding recovery, increasing efficacy of or decreasing resistance to alternative therapeutics, or a combination thereof. In some embodiments, “preventing” encompasses delaying the onset of symptoms, preventing relapse to a disease, decreasing the number or frequency of relapse episodes, increasing latency between symptomatic episodes, or a combination thereof. In some embodiments, “suppressing” or “inhibiting”, encompass reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

Thus, the anti-ATP6V1B2 antibodies disclosed herein would be useful in treating a disease associated with cellular senescence in a subject in need. As well, the anti-ATP6V1B2 antibodies disclosed herein would be useful as a follow-up treatment in a subject suffering from a cancer or tumor to prevent or reduce the incidence of recurrence or metastasis, or a combination thereof.

In some embodiments of method of treating a disease or condition associated with cellular senescence, the disease or condition associated with cellular senescence comprises a cancer or a tumor.

In some embodiments of a method of use for treating a disease or condition associated with cellular senescence, the antibody can be an IgG, a Fv, a scFv, a Fab, or a F(ab′)2. The IgG can be of the subclass of IgG1, IgG2, IgG3, or IgG4. In some embodiments of a method of use for treating a cancer or tumor, the antibody can be an IgG, a Fv, a scFv, a Fab, or a F(ab′)2. The IgG can be of the subclass of IgG1, IgG2, IgG3, or IgG4.

In some embodiments, a polynucleotide sequence encoding an anti-ATP6V1B2 antibody is used in a method of treating a subject with a disease or condition associated with cellular senescence, wherein the polynucleotide encodes an anti-ATP6V1B2 antibody as disclosed herein.

### Examples

### Example 1: Production and Analysis of Anti-hATP6V1B2 Antibodies

Objective: To produce and identify anti-hATP6V1B2 antibodies that recognize hATP6V1B2 on the surface of senescent cells.

### Methods & Results:

### Antibody Development

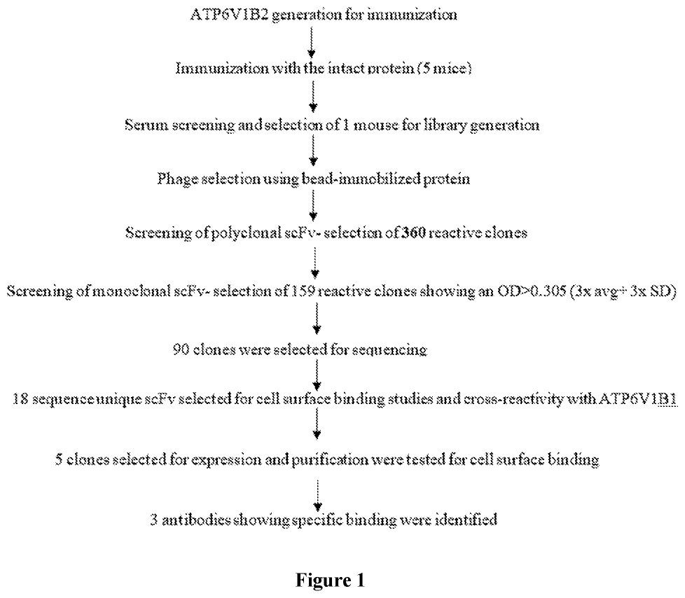

FIG. 1 presents a flow chart indicating the steps used for antibody development to make and identify monoclonal hybridoma clones that bind hATP6V1B2.

Human ATP6V1B2 protein was used for immunization. The protein was produced by acquiring the full-length human ATP6V1B2 sequence and generating clone constructs into a eukaryotic expression vector (ImmunoPrecise's-EU, ImmunoPrecise, Netherlands). Plasmid DNA was transiently expressed in HEK293 cells followed by harvesting, removal of supernatant, sonication, and purification.

Five mice were immunized with ATP6V1B2, followed by boosts (on days 22 and 42). Blood was withdrawn prior to immunization and on day 52 for serum screening using ELISA (Enzyme-Linked Immunosorbent Assay). ELISA was performed by coating 96-well plates with the recombinant protein (100 ng/well) overnight, blocking and subsequently incubating semi-log dilution series of sera. Serum reactivity was detected by incubating wells with HRP-conjugated anti-mouse-IgG secondary antibodies and TMB (3,3′,5,5′-Tetramethylbenzidine). Plates were read at 450 nm.

One mouse showing the highest reactivity profile towards ATP6V1B2 was selected for library generation. Splenocytes and bone marrow from the selected mouse were collected for RNA isolation followed by reverse transcription into cDNA. VH and VL genes were amplified using PCR and cloned into phagemid vector followed by electroporation in TG1 cells. Optimized versions of anti-ATP6V1B2 antibodies 2G05 and 2E11 were generated. DNA encoding anti-ATP6V1B2 antibodies 2G05 and 2E11 was synthesized and cloned into a vector containing mouse IgG2a harboring two mutations-S239D and 1332E (DE). The vector was used to transiently transfect 293T or Chinese hamster ovary (CHO) cells. Cell suspensions were collected and extracted using affinity chromatography. To obtain an afucosylated antibody, one of two methods was used: (1) for 293T, a decoy substrate which limits the incorporation of fucose during glycosylation; or (2) for CHO cells, fucose synthesis was redirected using a heterologous enzyme that depletes the fucose pool inside the cell (for CHO). Clone 4G06 was similarly optimized to include the two Fc mutations-S239D and 1332E (DE) and was produced as an afucosylated antibody (Data not shown).

Four rounds of phage selections on bead-immobilized recombinant protein (magnetic streptavidin beads or polystyrene magnetic beads) were performed. Polyclonal phage reactivity of 360 clones was assessed by ELISA resulting in the selection of 159 clones. Of these, 90 reactive clones were sent for sequencing. 18 sequence-unique clones were tested for binding to surface ATP6V1B2 in senescent and normal lung fibroblasts before selecting three clones for subsequent studies. Table 1 and Table 2 below present the Variable Heavy (VH) and Variable Light (VL) Chain CDR amino acid sequences, respectively.

Table 1
VH CDR1-CDR3 Amino Acid Sequences

| | | SEQ | | SEQ | | SEQ |
|---|---|---|---|---|---|---|
| Clone | | ID | | ID | | ID |
| ID | VH CDR1 | NO: | VH CDR2 | NO: | VH CDR3 | NO: |
| 2G05 | GYTFTSYW | 2 | IYPGSGST | 4 | AELGTFDY | 6 |
| 2E11 | GYTFTSYW | 2 | IYPGSGST | 4 | AELGTFDY | 6 |
| 4G06 | GYTFTNYW | 24 | IDPSDSDT | 26 | AELGTFDY | 6 |

TABLE 2
VL CDR1-CDR3 Amino Acid Sequences

| | | SEQ | | | SEQ |
|---|---|---|---|---|---|
| Clone | | ID | VL | | ID |
| ID | VL CDR1 | NO: | CDR2 | VL CDR3 | NO: |
| 2G05 | QSLVHSNGKTY | 9 | KVS | SQSTHVPYT | 13 |
| 2E11 | QSLVHSNGNTY | 20 | KVS | SQSTHVPYT | 13 |
| 4G06 | RSLVHSNGKTY | 29 | KVS | SQSTHVPWT | 31 |

Table 3 below presents the amino acid sequence of the VH and VL pairs for the three clones selected.

TABLE 3
Amino acid sequences of VH and VL domains

| | | SEQ | | SEQ |
|---|---|---|---|---|
| Clone | | ID | | ID |
| ID | VH | NO: | VL | NO: |
| 2G05 | EVQLQQSGPELVRPGA | 15 | DVVMTQTPLSLPVSLGD | 16 |
| | SVKISCKAPGYTFTSY | | QASISCRSSQSLVHSNG | |
| | WITWVKQRPGQGLEWI | | KTYLHWYLQKPGQSPKL | |
| | GDIYPGSGSTNYNEKF | | LIYKVSNRFSGVPDRFS | |
| | KSKATLTVDTSSSTAY | | GSGSGTDFTLKISRVEA | |
| | MQLSSLTSEDSAVYYC | | EDLGVYFCSQSTHVPYT | |
| | AELGTFDYWGQGTSLT | | FGGGTKLEIK | |
| | VST | | | |
| 2E11 | EVQLQQSGAELVKPGA | 21 | DVVVTQTPLSLPVSLGD | 22 |
| | SAKMSCRAPGYTFTSY | | QASISCRSSQSLVHSNG | |
| | WITWVKQRPGQGLEWI | | NTYLHWYLQKPGQSPKL | |
| | GDIYPGSGSTNYNEKF | | LIYKVSNRFSGVPDRFS | |
| | KTKATLTVDTSSSTAY | | GSGSGTDFTLKISRVEA | |
| | MQLSSLTSEDSAVYYC | | EDLGVYFCSQSTHVPYT | |
| | AELGTFDYWGQGTTLT | | FGGGTKLEIK | |
| | VST | | | |
| 4G06 | QVQLQQSGAELVKPGA | 32 | DIVMTQTPLSLPVSLGD | 33 |
| | SVKVSCKASGYTFTNY | | | |
| | WMHWVRQRPGQGLEWI | | QASIYCRSSRSLVHSNG | |
| | GRIDPSDSDTNYTQKF | | KTYLHWYQQKPGQSPKL | |
| | KGKATLTVDKSSNTAY | | LIYKVSNRFSGVPDRFS | |
| | MQVSSLTSEDFAVYYC | | GSGSGTDFTLKISRVEA | |
| | AELGTFDYWGQGTSLT | | EDLGVYFCSQSTHVPWT | |
| | VST | | FGGGTKLEIK | |

Table 4 and Table 5 below presents the amino acid sequences of the VH and VL Framework sequences for each clone, respectively.

TABLE 4
VH FR1-4 Amino Acid Sequences

| | | SEQ | | SEQ | | SEQ | | SEQ |
|---|---|---|---|---|---|---|---|---|
| Clone | | ID | | ID | | ID | VH | ID |
| ID | VH FR1 | NO: | VH FR2 | NO: | VH FR3 | NO: | FR4 | NO: |
| 2G05 | EVQLQQS | 1 | ITWVKQ | 3 | NYNEKFK | 5 | WGQGTS | 7 |
| | GPELVRP | | RPGQGL | | SKATLTV | | LTVST | |
| | GASVKIS | | EWIGD | | DTSSSTA | | | |
| | CKAP | | | | YMQLSSL | | | |
| | | | | | TSEDSAV | | | |
| | | | | | YYC | | | |
| 2E11 | EVQLQQS | 17 | ITWVKQ | 3 | NYNEKFK | 18 | WGQGTT | 34 |
| | GAELVKP | | RPGQGL | | TKATLTV | | LTVST | |
| | GASAKMS | | EWIGD | | DTSSSTA | | | |
| | CRAP | | | | YMQLSSL | | | |
| | | | | | TSEDSAV | | | |
| | | | | | YYC | | | |
| 4G06 | QVQLQQS | 23 | MHWVRQ | 25 | NYTQKFK | 27 | WGQGTS | 7 |
| | GAELVKP | | RPGQGL | | GKATLTV | | LTVST | |
| | GASVKVS | | EWIGR | | DKSSNTA | | | |
| | CKAS | | | | YMQVSSL | | | |
| | | | | | TSEDFAV | | | |
| | | | | | YYC | | | |

TABLE 5
VL FR1-4 Amino Acid Sequences

| | | SEQ | | SEQ | | SEQ | | SEQ |
|---|---|---|---|---|---|---|---|---|
| Clone | | ID | | ID | | ID | VL | ID |
| ID | VL FR1 | NO: | VL FR2 | NO: | VL FR3 | NO: | R4 | NO: |
| 2G05 | DVVMTQT | 8 | LHWYLQ | 10 | NRFSGVP | 12 | FGGG | 14 |
| | PLSLPVS | | KPGQSP | | DRFSGSG | | TKLE | |
| | LGDQASI | | KLLIY | | SGTDFTL | | IK | |
| | SCRSS | | | | KISRVEA | | | |
| | | | | | EDLGVYF | | | |
| | | | | | C | | | |
| 2E11 | DVVVTQT | 19 | LHWYLQ | 10 | NRFSGVP | 12 | FGGG | 14 |
| | PLSLPVS | | KPGQSP | | DRFSGSG | | TKLE | |
| | LGDQASI | | KLLIY | | SGTDFTL | | IK | |
| | SCRSS | | | | KISRVEA | | | |
| | | | | | EDLGVYF | | | |
| | | | | | C | | | |
| 4G06 | DIVMTQT | 28 | LHWYQQ | 30 | NRFSGVP | 12 | FGGG | 14 |
| | PLSLPVS | | KPGQSP | | DRFSGSG | | TKLE | |
| | GLDQASI | | KLLIY | | SGTDFTL | | IK | |
| | YCRSS | | | | KISRVEA | | | |
| | | | | | EDLGVYF | | | |
| | | | | | C | | | |

### ELISA Screening of Clones for ATP6V1B2 or ATP6V1B1 Binding

ELISA was performed by coating 96-well plates with the recombinant protein: ATP6V1B2 or ATP6V1B1 (1 μg/ml) and incubating the plate overnight, blocking (350 μl/well of 1% BSA/PBS) for 60 minutes at room temperature and subsequently incubating with periplasmic dilution of 1:4 for 90 minutes at room temperature. Proteins were detected by incubating wells with HRP-conjugated anti-mouse-IgG secondary antibodies and TMB (3,3′,5,5′-Tetramethylbenzidine). Plates were read at 450 nm.

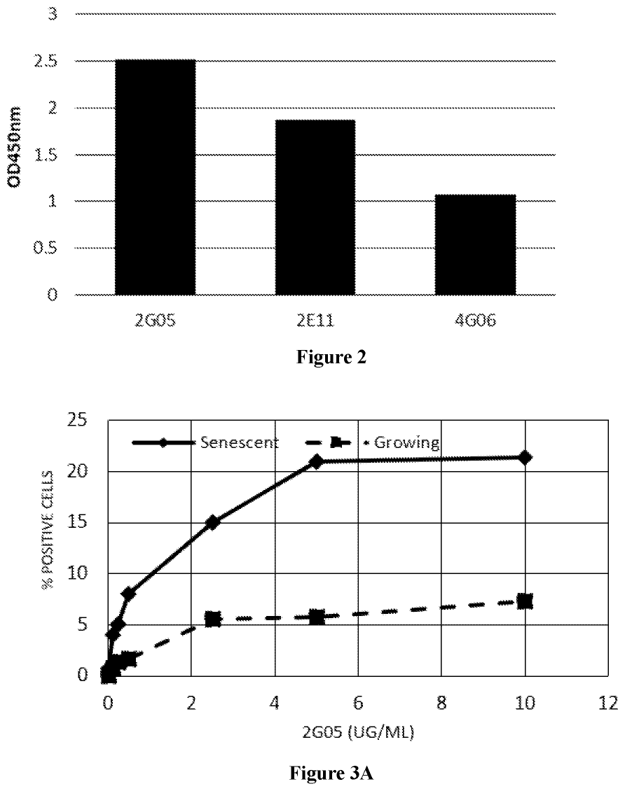

FIG. 2 shows that three anti-ATP6V1B2 antibodies (2G05, 2E11 and 4G06) bind to human recombinant protein. Clones were tested as short chain variable fragments (scFv) using ELISA.

### Binding of 2G05, 2E11 AND 4G06 or Commercial Anti-ATP6V1B2 Antibodies to Senescent Human and Mouse Lung Fibroblasts

Senescence was induced in IMR-90 (human lung fibroblasts) or CCL-206 (mouse lung fibroblasts) by a 48h incubation with etoposide (100 uM for IMR-90, 35 uM for CCL-206) followed by a 7-12-day incubation in culture medium. Control, growing IMR-90 or CCL-206 cells were seeded 24h prior to testing day. On testing day, the medium was aspirated, and cells were gently detached using warm TrypLE Express solution (Thermo Fisher Scientific) followed by the addition of cold FACS buffer (PBS/5% FBS). ATP6V1B2 antibodies were added to the tubes containing the cells and the tubes were incubated for 60-70 minutes at 4C and centrifuged. Antibodies 2G05, 2E11 and 4G06 were tested at 10 ug/ml. Commercial antibodies-LS-B13421 (LS Bio), ab200839 (Abcam), and SC-55544 (Santa Cruz) were used at 20 ug/ml. Two hundred fifty ul of secondary antibody (anti mouse Alexa 647, Jackson 115-605-146 diluted in FACS buffer 1:300) were added and the tubes were incubated for 40 minutes at 4 C. Following incubation, 600 ul FACS buffer supplemented with DAPI (diluted 1:20,000) were added to each tube and the tubes were centrifuged. Cell pellets were resuspended in 200 ul FACS buffer. The samples were analyzed using GUAVA Flow Cytometry analyzer. Data was analyzed using the FCSalyzer 0.9.18-alpha software. Duplicates and dead cells (DAPI positive cells) were excluded.

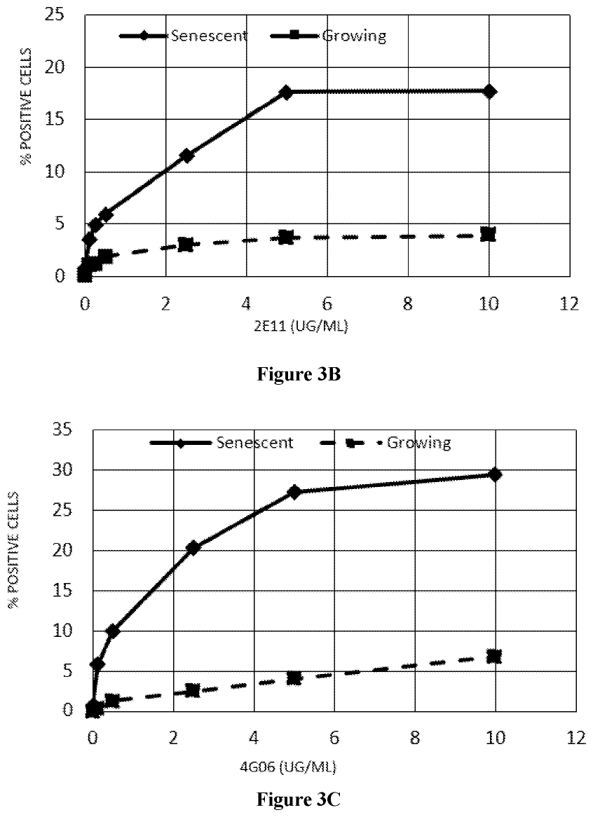

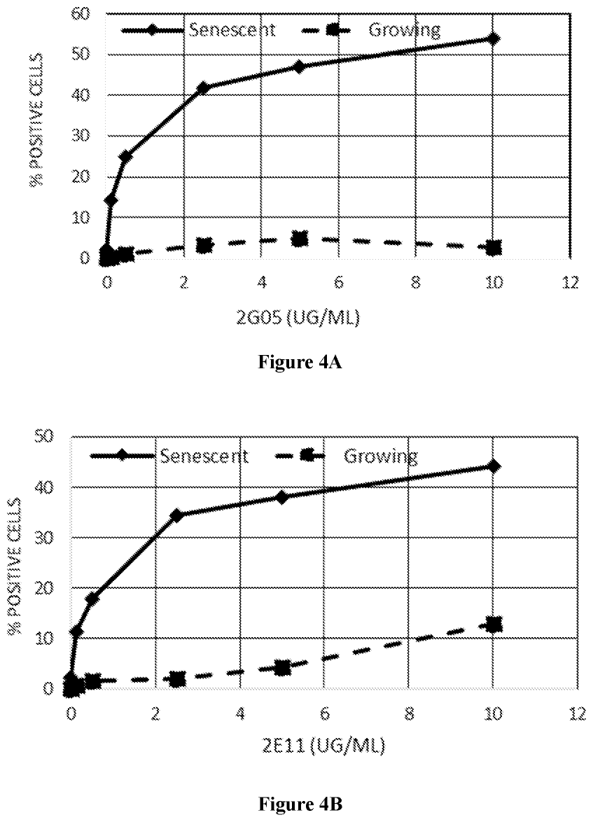

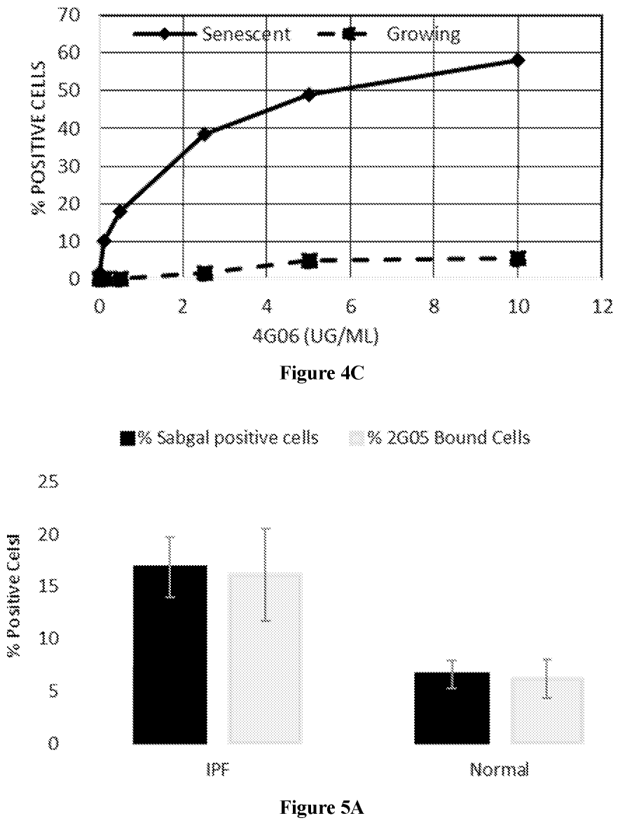

FIGS. 3A-3C show that the three anti-ATP6V1B2 antibodies (2G05, 2E11 and 4G06) preferentially bind the surface protein expressed in senescent human lung fibroblasts (IMR-90). FIGS. 4A-4C show that the three anti-ATP6V1B2 antibodies (2G05, 2E11 and 4G06) preferentially bind the surface protein expressed in senescent mouse lung fibroblasts (CCL-206).

### Binding of 2G05 and 2E11 to Human Idiopathic Pulmonary Fibrosis (IPF) Fibroblasts

IPF lung fibroblasts or normal lung fibroblasts were obtained from Lonza (catalog number CC-7231 and CC-2512, respectively). Cells were used at passages 3-6. On testing day, the medium was aspirated, and cells were gently detached using warm TrypLE Express solution (Thermo Fisher Scientific) followed by the addition of cold FACS buffer (PBS/5% FBS). ATP6V1B2 antibodies were added to the tubes containing the cells and the tubes were incubated for 60-70 minutes at 4C and centrifuged. Two hundred fifty ul of secondary antibody (anti mouse Alexa 647, Jackson 115-605-146 diluted in FACS buffer 1:300) were added and the tubes were incubated for 40 minutes at 4 C. Following incubation, 600 ul FACS buffer supplemented with DAPI (diluted 1:20,000) were added to each tube and the tubes were centrifuged. Cell pellets were resuspended in 200 ul FACS buffer. The samples were analyzed using GUAVA Flow Cytometry analyzer. For SA beta gal staining, culture plates were washed with PBS, and the cells were fixed with 0.5% Glutaraldehyde solution (in PBS pH7.4) for 15 min at room temperature (RT). Cells were then washed with PBS for 5 minutes followed by addition of a solution of PBS/MgCl₂, pH 6.0 (twice, 5 minutes each, RT). Freshly prepared X-Gal staining solution was then added, and the plates were incubated overnight in a 37C incubator (with no CO₂). Following incubation, the plates were washed 3 times with PBS and inspected by a light microscope. Senescence quantification was conducted using a light microscope by counting stained cells in 20 different fields.

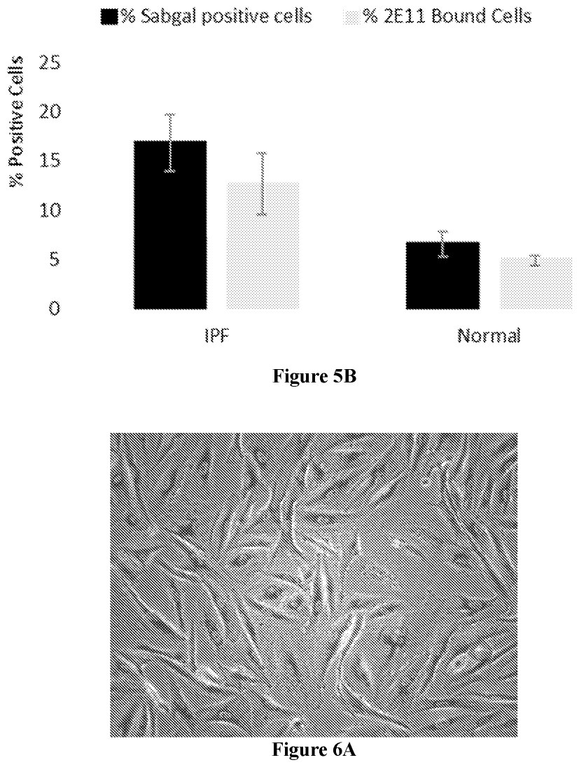

FIGS. 5A-5B show that antibody 2G05 and antibody 2E11 bind to senescent IPF (Idiopathic Pulmonary Fibrosis) lung fibroblasts.

### Binding of 2G05 and 2E11 to Senescent Tumor Cells

Senescence was induced in A549 lung adenocarcinoma cells by incubating them with 100 uM doxorubicin for 7 days. Control, growing cells were seeded in 10 cm plates a day prior to testing. For SA-beta gal staining cells were fixed with 0.5% Glutaraldehyde solution (in PBS pH7.4) for 15 min at RT followed by 5-minute washing with PBS. Cells were then washed x2 with PBS/MgCl₂ pH 6.0 for 5 min at RT. Freshly prepared X-gal solution was added, and the plates were incubated at 37C (sealed, protected from light and in an incubator without CO₂) for 16 h. The plates were washed x3 with PBS for 5 min at RT. Pictures were taken at this point.

To determine cell surface binding, the cells were gently detached from the plates using TrypLE Express (1.5 ml per plate, 5-10 minutes incubation). FACS buffer (5% FBS in PBS) was then added to the plates and the cells were collected into 15 ml tubes and kept on ice. Tubes were centrifuged at 200×g and 4C for 8 min. Pellets were re-suspended in FACS buffer and distributed at equal volumes into 1.5 ml tubes. Following centrifugation, primary 5 ug/ml 2E11 or 2G05 were added for an incubation of 60-70 minutes at 4 C. Six hundred ul FACS buffer were then added, and the tubes were centrifuged. Following centrifugation, secondary antibody was added (anti mouse Alexa 647) for 40 minutes at 4 C. Tubes were protected from light. Six hundred ul FACS buffer supplemented with DAPI (diluted 1:30,000) was added to each tube. Pellets were resuspended with 200 ul FACS buffer. Samples were analyzed using GUAVA Flow Cytometry Analyzer. Data was analyzed using the FCSalyzer 0.9.18-alpha software. Duplicates and dead cells (DAPI positive) were excluded.

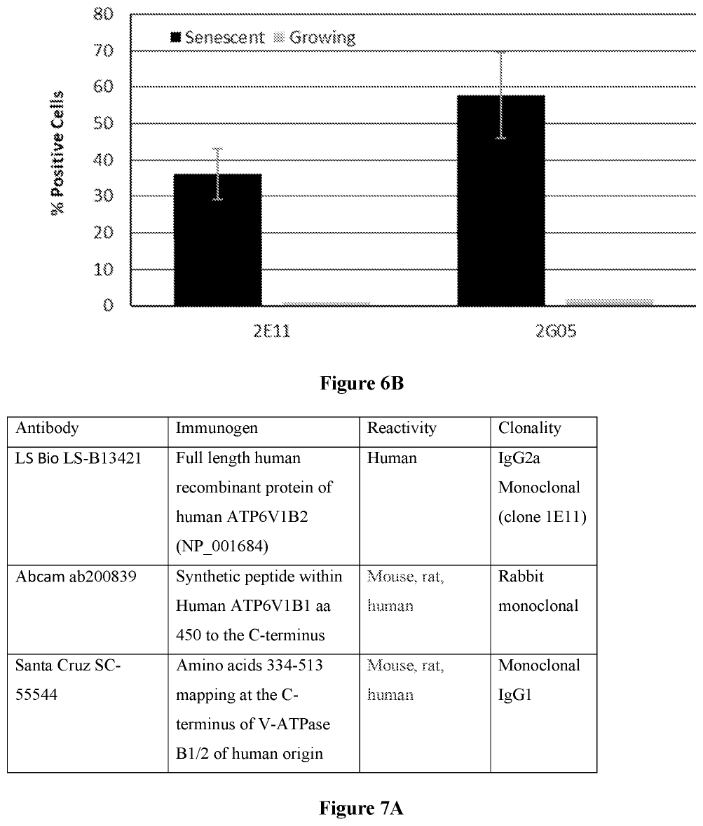

FIGS. 6A-6B show anti-ATP6V1B2 antibodies 2G05 or 2E11 bind to senescent A549 lung adenocarcinoma cells but not to control non-senescent A549 cells.

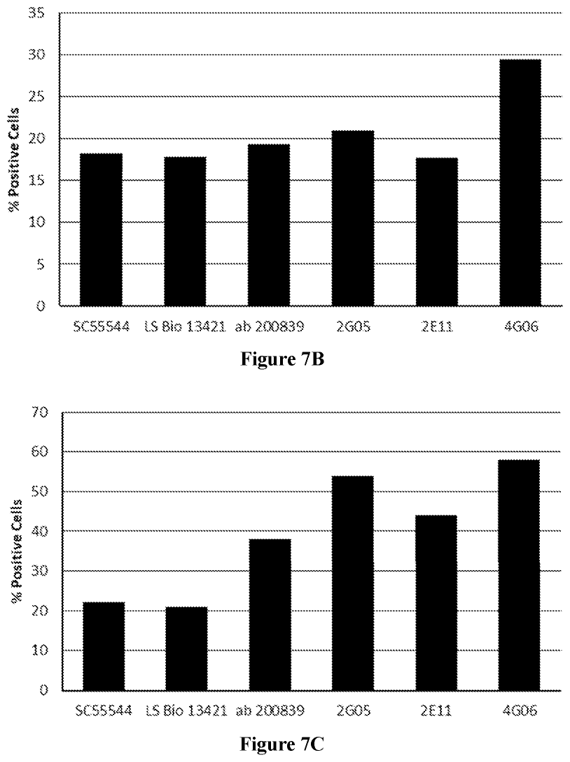

FIGS. 7A-7C show binding of the three anti-ATP6V1B2 antibodies (2G05, 2E11 and 4G06) to senescent IMR-90 or CCL-206 cells as compared with three commercially available anti-ATP6V1B2 antibodies.

### Antibody Sequences

### 2G05

Anti-ATP6V1B2 antibody 2G05 comprises a heavy chain variable region (VH) comprising complementarity determining region 1 (HCDR1), HCDR2 and HCDR3, and a light chain variable region (VL) having complementarity determining region 1 (LCDR1), LCDR2 and LCDR3, wherein the HCDR1, HCDR2 and HCDR3 comprise the amino acid sequences of SEQ ID NOs: 2, 4, and 6, respectively, and the LCDR1, LCDR2 and LCDR3 comprise the amino acid sequences of SEQ ID NO: 9, amino acids KVS, and SEQ ID NO: 13, respectively. The VH and VL of antibody 2G05 comprise the amino acid sequences of SEQ ID NOs: 15 and 16 respectively.

### 2E11

Anti-ATP6V1B2 antibody 2E11 comprises a heavy chain variable region (VH) comprising complementarity determining region 1 (HCDR1), HCDR2 and HCDR3, and a light chain variable region (VL) having complementarity determining region 1 (LCDR1), LCDR2 and LCDR3, wherein the HCDR1, HCDR2 and HCDR3 comprise the amino acid sequences of SEQ ID NOs: 2, 4, and 6, respectively, and the LCDR1, LCDR2 and LCDR3 comprise the amino acid sequences of SEQ ID NO: 20, amino acids KVS, and SEQ ID NO: 13, respectively. The VH and VL of antibody 2E11 comprise the amino acid sequences of SEQ ID NOs: 21 and 22 respectively.

### 4G06

Anti-ATP6V1B2 antibody 4G06 comprises a heavy chain variable region (VH) comprising complementarity determining region 1 (HCDR1), HCDR2 and HCDR3, and a light chain variable region (VL) having complementarity determining region 1 (LCDR1), LCDR2 and LCDR3, wherein the HCDR1, HCDR2 and HCDR3 comprise the amino acid sequences of SEQ ID NOs: 24, 26, and 6, respectively, and the LCDR1, LCDR2 and LCDR3 comprise the amino acid sequences of SEQ ID NO: 29, amino acids KVS, and SEQ ID NO: 31, respectively. The VH and VL of antibody 4G06 comprise the amino acid sequences of SEQ ID NOs: 32 and 33 respectively.

### Example 2: Antibody Dependent Cell Mediated Cytotoxicity (ADCC) Activity of Anti-ATP6V1B2 Antibodies

Objective: To Characterize the antibodies by analyzing Antibody Dependent Cell Mediated Cytotoxicity (ADCC) Activity

Methods and Results: Senescent CCL-206 cells were used as target cells. Senescence was induced in CCL-206 cells by a 48h incubation with 35 uM etoposide followed by 24h incubation in culture medium. Plates were then washed with PBS and the cells were gently detached using warm TrypLE Express. Cells were seeded in flat bottom 96-well plate and are incubated for 7-14 days. ADCC activity was tested using the mFcγRIV ADCC Reporter Bioassay (Promega M1211) according to manufacturer's instructions. On assay day, medium was aspirated and the tested antibodies (2E11, 2G05 or IgG2a control were added). Following a 20-minute incubation, effector cells were added to each of the wells containing target cells and antibodies at an effector: target cell ratio of 10:1. Plates were covered and incubated in a 37C, 5% CO₂ incubator for 6 hrs. Bio-Glo Reagent were added to each well and the plates were incubated for 5-30 minutes. Luminescence was measured using a plate reader (CLARIOstar plate reader BMG LABTECH).

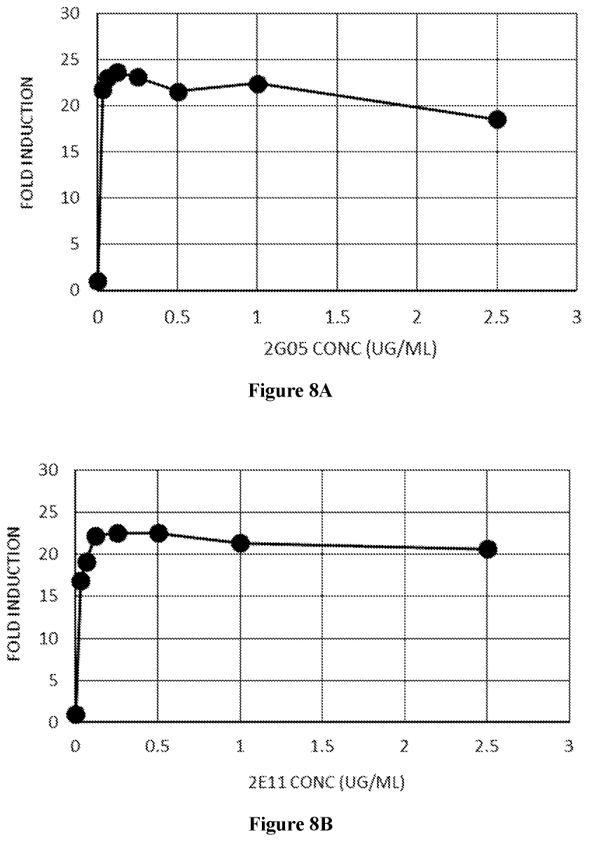

FIGS. 8A-8B show anti-ATP6V1B2 antibodies 2G05 and 2E11 activate the ADCC pathway. Antibodies 2G05 and 2E11 are formatted as IgG2a comprising two substation mutations in the Fc region (S239D and 1332E) and were produced in the absence of fucose. Clone 4G06 was similarly formatted as an IgG2a comprising two substation mutations in the Fc region (S239D and 1332E) and were produced in the absence of fucose (data not shown).

### Example 3: Cross Reactivity with ATP6V1B1

Objective: To assay the antibodies for cross-reactivity with ATP6V1B1.

### Methods & Results:

FIG. 9 shows two anti-ATP6V1B2 antibodies (2G05, 4G06) have cross reactivity with ATP6V1B1 complex. The antibodies were tested as short chain variable fragments (scFv) using ELISA.

The ability of these antibodies to bind to both subunits should increase the affinity of the antibody to the whole ATPase complex, owing that two different subunits are recognized, and two molecules of the antibody can potentially bind one ATPase complex. The cross-reactivity of these antibodies increases the potential of the antibody to target senescent cells.

While certain features of the anti-ATP6V1B2 antibodies and there uses have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. An isolated anti-ATP6V1B2 antibody, wherein the antibody comprises a heavy chain variable region (VH) comprising complementarity determining region 1 (HCDR1), HCDR2 and HCDR3, and a light chain variable region (VL) having complementarity determining region 1 (LCDR1), LCDR2 and LCDR3,
wherein said HCDR1, HCDR2 and HCDR3 and said LCDR1, LCDR2 and LCDR3 for the antibody comprise the amino acid sequences of
(i) SEQ ID NOs: 2, 4, and 6, respectively, and SEQ ID NO: 9, KVS, and SEQ ID NO: 13, respectively; or
(ii) SEQ ID NOs: 2, 4, and 6, respectively, and SEQ ID Ns: 20, KVS, and SEQ ID NO: 13 respectively; or
(iii) SEQ ID NOs: 24, 26, and 6, respectively, and SEQ ID NOs: 29, KVS, and SEQ ID NO: 31, respectively.

2. The antibody of claim 1, further comprising a heavy chain fragment crystallizable region (Fc region), wherein said Fc region comprises at least one amino acid residue substitution comprising S239D, 1332E, A330L, G236A, H268F, S324T, or S267E, or any combination thereof; or wherein fucosylation of the Fc region is reduced in comparison to a Fc region of an anti-ATP6V1B2 antibody produced in the presence of fucose; or both.

3. The antibody of claim 2, further comprising a Fc region comprising at least one amino acid residue substitution combinations comprising S239D/1332E, A330L/S239D/1332E, G236A/1332E, H268F/S324T, S267E/H268F/S324T, or any combination thereof.

4. The antibody of claim 2, wherein the Fc region is afucosylated.

5. The antibody of claim 2, wherein binding of the anti-ATP6V1B2 antibody comprising the at least one amino acid residue substitution to an ATP6V1B2 epitope moiety enhances antibody-dependent cell-mediated cytotoxicity (ADCC), complement dependent cytotoxicity (CDC), antibody-dependent cellular phagocytosis (ADCP), or any combination thereof in comparison with binding of a wild-type anti-ATP6V1B2 antibody to the ATP6V1B2 epitope moiety.

6. The antibody of claim 2, wherein binding of the anti-ATP6V1B2 antibody to an ATP6V1B2 epitope moiety enhances antibody-dependent cell-mediated cytotoxicity (ADCC), complement dependent cytotoxicity (CDC), antibody-dependent cellular phagocytosis (ADCP), or any combination thereof in comparison with binding of an anti-ATP6V1B2 antibody produced in the presence of fucose to the ATP6V1B2 epitope moiety.

7. The antibody of claim 1, wherein said VH and VL comprise the amino acid sequences of SEQ ID NOs: 15 and 16, or SEQ ID NOs: 21 and 22, or SEQ ID NOs: 32 and 33.

8. The antibody of claim 2, wherein the antibody comprises an IgG, a Fv, a scFv, a Fab, or a F(ab′)2.

9. The antibody of claim 1, wherein the amino acid sequence of said heavy chain variable region comprises one or more humanized framework (FR) sequences and the amino acid sequence of said light chain variable region comprises one or more humanized FR sequences.

10. A composition comprising the isolated antibody of claim 1 and a pharmaceutically acceptable carrier.

11. A nucleic acid construct comprising one or more nucleic acid sequences, said nucleic acid sequences encoding a light chain variable region (VL) and a heavy chain variable region (VH), of the anti-ATP6V1B2 antibody of claim 1.

12. An expression vector comprising the nucleic acid construct of claim 11.

13. A host cell comprising the expression vector of claim 12.

14. A method for treating a disease associated with cellular senescence comprising administering a composition comprising an effective amount of the anti-ATP6V1B2 antibody of claim 1, wherein:
(a) the anti-ATP6V1B2 antibody further comprises a heavy chain fragment crystallizable region (Fc region) comprising at least one amino acid residue substitution comprising S239D, 1332E, A330L, G236A, H268F, S324T, S267E, or any combination thereof; or wherein fucosylation of the Fc region is reduced in comparison to a Fc region of an anti-ATP6V1B2 antibody produced in the presence of fucose; or both;
(b) the anti-ATP6V1B2 antibody further comprises an ATP6V1B2 antibody-drug conjugate; or
(c) both (a) and (b).

15. The method of claim 14, wherein said at least one amino acid residue substitution comprises two substitutions S239D and 1332E, and fucosylation of the Fc region is reduced in comparison to a Fc region of an anti-ATP6V1B2 antibody produced in the presence of fucose.

16. (canceled)

17. The method of claim 14, wherein said disease associated with cellular senescence is an age-related disease or condition.


SEQUENCE LISTING (34 sequences)

SEQ ID NO: 1 - protein, 25 aa, synthetic construct
  REGION 1..25: note = Description of Artificial Sequence: Synthetic peptide
EVQLQQSGPELVRPGASVKISCKAP

SEQ ID NO: 2 - protein, 8 aa, synthetic construct
  REGION 1..8: note = Description of Artificial Sequence: Synthetic peptide
GYTFTSYW

SEQ ID NO: 3 - protein, 17 aa, synthetic construct
  REGION 1..17: note = Description of Artificial Sequence: Synthetic peptide
ITWVKQRPGQGLEWIGD

SEQ ID NO: 4 - protein, 8 aa, synthetic construct
  REGION 1..8: note = Description of Artificial Sequence: Synthetic peptide
IYPGSGST

SEQ ID NO: 5 - protein, 38 aa, synthetic construct
  REGION 1..38: note = Description of Artificial Sequence: Synthetic polypeptide
NYNEKFKSKATLTVDTSSSTAYMQLSSLTSEDSAVYYC

SEQ ID NO: 6 - protein, 8 aa, synthetic construct
  REGION 1..8: note = Description of Artificial Sequence: Synthetic peptide
AELGTFDY

SEQ ID NO: 7 - protein, 11 aa, synthetic construct
  REGION 1..11: note = Description of Artificial Sequence: Synthetic peptide
WGQGTSLTVSV

SEQ ID NO: 8 - protein, 26 aa, synthetic construct
  REGION 1..26: note = Description of Artificial Sequence: Synthetic peptide
DVVMTQTPLSLPVSLGDQASISCRSS

SEQ ID NO: 9 - protein, 11 aa, synthetic construct
  REGION 1..11: note = Description of Artificial Sequence: Synthetic peptide
QSLVHSNGKTY

SEQ ID NO: 10 - protein, 17 aa, synthetic construct
  REGION 1..17: note = Description of Artificial Sequence: Synthetic peptide
LHWYLQKPGQSPKLLIY

SEQ ID NO: 11 - skipped

SEQ ID NO: 12 - protein, 36 aa, synthetic construct [sequence omitted]

SEQ ID NO: 13 - protein, 9 aa, synthetic construct
  REGION 1..9: note = Description of Artificial Sequence: Synthetic peptide
SQSTHVPYT

SEQ ID NO: 14 - protein, 10 aa, synthetic construct
  REGION 1..10: note = Description of Artificial Sequence: Synthetic peptide
FGGGTKLEIK

SEQ ID NO: 15 - protein, 115 aa, synthetic construct
  REGION 1..115: note = Description of Artificial Sequence: Synthetic polypeptide
EVQLQQSGPELVRPGASVKISCKAPGYTFTSYWITWVKQRPGQGLEWIGDIYPGSGSTNYNEKFKSKATLTVDTSSSTAYMQLSSLTSEDSAVYYCAELGTFDYWGQGTSLTVSV

SEQ ID NO: 16 - protein, 112 aa, synthetic construct
  REGION 1..112: note = Description of Artificial Sequence: Synthetic polypeptide
DVVMTQTPLSLPVSLGDQASISCRSSQSLVHSNGKTYLHWYLQKPGQSPKLLIYKVSNRFSGVPDRFSGSGSGTDFTLKISRVEAEDLGVYFCSQSTHVPYTFGGGTKLEIK

SEQ ID NO: 17 - protein, 25 aa, synthetic construct
  REGION 1..25: note = Description of Artificial Sequence: Synthetic peptide
EVQLQQSGAELVKPGASAKMSCRAP

SEQ ID NO: 18 - protein, 38 aa, synthetic construct
  REGION 1..38: note = Description of Artificial Sequence: Synthetic polypeptide
NYNEKFKTKATLTVDTSSSTAYMQLSSLTSEDSAVYYC

SEQ ID NO: 19 - protein, 26 aa, synthetic construct
  REGION 1..26: note = Description of Artificial Sequence: Synthetic peptide
DVVVTQTPLSLPVSLGDQASISCRSS

SEQ ID NO: 20 - protein, 11 aa, synthetic construct
  REGION 1..11: note = Description of Artificial Sequence: Synthetic peptide
QSLVHSNGNTY

SEQ ID NO: 21 - protein, 115 aa, synthetic construct
  REGION 1..115: note = Description of Artificial Sequence: Synthetic polypeptide
EVQLQQSGAELVKPGASAKMSCRAPGYTFTSYWITWVKQRPGQGLEWIGDIYPGSGSTNYNEKFKTKATLTVDTSSSTAYMQLSSLTSEDSAVYYCAELGTFDYWGQGTTLTVST

SEQ ID NO: 22 - protein, 112 aa, synthetic construct
  REGION 1..112: note = Description of Artificial Sequence: Synthetic polypeptide
DVVVTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIK

SEQ ID NO: 23 - protein, 25 aa, synthetic construct
  REGION 1..25: note = Description of Artificial Sequence: Synthetic peptide
QVQLQQSGAELVKPGASVKVSCKAS

SEQ ID NO: 24 - protein, 8 aa, synthetic construct
  REGION 1..8: note = Description of Artificial Sequence: Synthetic peptide
GYTFTNYW

SEQ ID NO: 25 - protein, 17 aa, synthetic construct
  REGION 1..17: note = Description of Artificial Sequence: Synthetic peptide
MHWVRQRPGQGLEWIGR

SEQ ID NO: 26 - protein, 8 aa, synthetic construct
  REGION 1..8: note = Description of Artificial Sequence: Synthetic peptide
IDPSDSDT

SEQ ID NO: 27 - protein, 38 aa, synthetic construct
  REGION 1..38: note = Description of Artificial Sequence: Synthetic polypeptide
NYTQKFKGKATLTVDKSSNTAYMQVSSLTSEDFAVYYC

SEQ ID NO: 28 - protein, 26 aa, synthetic construct
  REGION 1..26: note = Description of Artificial Sequence: Synthetic peptide
DIVMTQTPLSLPVSLGDQASIYCRSS

SEQ ID NO: 29 - protein, 11 aa, synthetic construct
  REGION 1..11: note = Description of Artificial Sequence: Synthetic peptide
RSLVHSNGKTY

SEQ ID NO: 30 - protein, 17 aa, synthetic construct
  REGION 1..17: note = Description of Artificial Sequence: Synthetic peptide
LHWYQQKPGQSPKLLIY

SEQ ID NO: 31 - protein, 9 aa, synthetic construct
  REGION 1..9: note = Description of Artificial Sequence: Synthetic peptide
SQSTHVPWT

SEQ ID NO: 32 - protein, 115 aa, synthetic construct
  REGION 1..115: note = Description of Artificial Sequence: Synthetic polypeptide
QVQLQQSGAELVKPGASVKVSCKASGYTFTNYWMHWVRQRPGQGLEWIGRIDPSDSDTNYTQKFKGKATLTVDKSSNTAYMQVSSLTSEDFAVYYCAELGTFDYWGQGTSLTVSV

SEQ ID NO: 33 - protein, 112 aa, synthetic construct
  REGION 1..112: note = Description of Artificial Sequence: Synthetic polypeptide
DIVMTQTPLSLPVSLGDQASIYCRSSRSLVHSNGKTYLHWYQQKPGQSPKLLIYKVSNRFSGVPDRFSGSGSGTDFTLKISRVEAEDLGVYFCSQSTHVPWTFGGGTKLEIK

SEQ ID NO: 34 - protein, 11 aa, synthetic construct
WGQGTTLTVST